(19) United States
(12) Patent Application Publication
(10) Pub. No.: US 2024/0285664 A1
(43) Pub. Date: Aug. 29, 2024

(54) LINE-1 INHIBITORS AS COGNITIVE ENHANCERS

(71) Applicant: TRANSPOSON THERAPEUTICS, INC., San Diego, CA (US)

(72) Inventors: Eckard WEBER, Los Angeles, CA (US); Andrew SATLIN, New York, NY (US)

(21) Appl. No.: 18/566,413

(22) PCT Filed: Jun. 3, 2022
(86) PCT No.: PCT/US2022/032120

§ 371 Date: Dec. 1, 2023

Related U.S. Application Data
(60) Provisional application No. 63/197,015, filed on Jun. 4, 2021.

(51) Int. Cl. A61K 31/7076; A61K 31/7072

(52) U.S. Cl. CPC A61K 31/7076; A61K 31/7072

(57) ABSTRACT

The present disclosure provides methods of enhancing cognition, inhibiting cognitive decline, treating or preventing a cognitive deficit disorder, or treating or preventing Creutzfeldt-Jakob disease (CJD) in a subject in need thereof comprising administering a LINE-1 inhibitor, or a pharmaceutical composition thereof, to the subject.

### BACKGROUND OF THE INVENTION

### Field of the Invention

The present disclosure provides methods of enhancing cognition, inhibiting cognitive decline, treating or preventing a cognitive deficit disorder, or treating or preventing Creutzfeldt-Jakob disease (CID) in a subject in need thereof comprising administering a LINE-1 inhibitor, or a pharmaceutical composition thereof, to the subject.

### Background

Research suggests that the level of protein kinase R (PKR) is involved in the memory/cognitive defects associated with Alzheimer's disease. See, e.g., Hugon et al., Alzheimer's Research & Therapy 9:83 (2017). https://doi.org/10.1186/s13195-017-0308-0. There exists a need in the art for new cognitive-enhancing drugs. See, e.g., Mehlman, Milbank Q. 82(3):483-506 (2004). https://doi.org/10.1111/j.0887-378X.2004.00319.x.

### BRIEF SUMMARY OF THE INVENTION

In one aspect, the present disclosure provides methods of enhancing cognition, inhibiting cognitive decline, or treating or preventing a cognitive deficit disorder in a subject in need thereof with a therapeutically effective amount of a LINE-1 inhibitor. Exemplary LINE-1 inhibitors include, but are not limited to, islatravir, censavudine, and elvucitabine.

In another aspect, the present disclosure provides a LINE-1 inhibitor for use in enhancing cognition, inhibiting cognitive decline, or treating or preventing a cognitive deficit disorder in a subject in need thereof.

In another aspect, the present disclosure provides the use of a LINE-1 inhibitor for the manufacture of a medicament for enhancing cognition, inhibiting cognitive decline, or treating or preventing a cognitive deficit disorder in a subject in need thereof.

In another aspect, the present disclosure provides a kit comprising a LINE-1 inhibitor, or a pharmaceutical composition thereof, and instructions of administering the LINE-1 inhibitor, or pharmaceutical composition thereof, to enhance cognition, inhibit cognitive decline, or treat or prevent a cognitive deficit disorder in a subject in need thereof.

In another aspect, the present disclosure provides methods of treating or preventing CJD in a subject in need thereof with a therapeutically effective amount of a LINE-1 inhibitor. Exemplary LINE-1 inhibitors include, but are not limited to, islatravir, censavudine, and elvucitabine.

In another aspect, the present disclosure provides a LINE-1 inhibitor for use in treating or preventing CID in a subject in need thereof.

In another aspect, the present disclosure provides the use of a LINE-1 inhibitor for the manufacture of a medicament for treating or preventing CID in a subject in need thereof.

In another aspect, the present disclosure provides a kit comprising a LINE-1 inhibitor, or a pharmaceutical composition thereof, and instructions of administering the LINE-1 inhibitor, or pharmaceutical composition thereof, to treat or prevent CJD in a subject in need thereof.

### DETAILED DESCRIPTION OF THE INVENTION

### I. Therapeutic Methods and Uses

It has been unexpectedly discovered that LINE-1 inhibitors indirectly inhibit the expression of PKR and thus can be used a cognition-enhancing drugs.

In one embodiment, the disclosure provides a method of enhancing cognition, inhibiting cognitive decline, or treating or preventing a cognitive deficit disorder in a subject in need thereof, the method comprising administering a therapeutically effective amount of a LINE-1 inhibitor to the subject. A LINE-1 inhibitor can be administered to any subject in need of cognitive enhancement, e.g., in cognitively healthy subject or a subject with a cognitive deficit.

In another embodiment, the disclosure provides a LINE-1 inhibitor for use in enhancing cognition, inhibiting cognitive decline, or treating or preventing a cognitive deficit disorder in a subject.

In another embodiment, the disclosure provides the use of a LINE-1 inhibitor in the manufacture of a medicament for enhancing cognition, inhibiting cognitive decline, or treating or preventing a cognitive deficit disorder.

In one embodiment, the disclosure provides a method of treating or preventing CJD in a subject in need thereof, the method comprising administering a therapeutically effective amount of a LINE-1 inhibitor to the subject.

In another embodiment, the disclosure provides a LINE-1 inhibitor for use in treating or preventing CID in a subject.

In another embodiment, the disclosure provides the use of a LINE-1 inhibitor in the manufacture of a medicament for treating or preventing CID.

In another embodiment, the subject is (a) not infected with the HIV virus, (b) is not suspected of being infected with the HIV virus, and/or (c) is not being treated to prevent infection with the HIV virus.

In another embodiment, the subject (a) does not have Alzheimer's disease, (b) is not suspected of having or been diagnosed as having Alzheimer's disease; and/or (c) is not being treated for Alzheimer's disease.

In another embodiment, the subject (a) does not have Parkinson's disease, (b) is not suspected of having or been diagnosed as having Parkinson's disease; and/or (c) is not being treated for Parkinson's disease.

In another embodiment, the subject (a) does not have Huntington's disease, (b) is not suspected of having or been diagnosed as having Huntington's disease; and/or (c) is not being treated for Huntington's disease.

In another embodiment, the subject (a) does not have frontotemporal dementia, (b) is not suspected of having or been diagnosed as having frontotemporal dementia; and/or (c) is not being treated for frontotemporal dementia.

In another embodiment, the subject (a) does not have multiple sclerosis, (b) is not suspected of having or been diagnosed as having multiple sclerosis; and/or (c) is not being treated for multiple sclerosis.

In another embodiment, the subject (a) does not have Aicardi Goutiere's syndrome, (b) is not suspected of having or been diagnosed as having Aicardi Goutiere's syndrome; and/or (c) is not being treated for Aicardi Goutiere's syndrome.

In another embodiment, the subject (a) does not have progressive supra nuclear palsy, (b) is not suspected of having or been diagnosed as having progressive supra nuclear palsy; and/or (c) is not being treated for progressive supra nuclear palsy.

In another embodiment, the subject (a) does not have schizophrenia, (b) is not suspected of having or been diagnosed as having schizophrenia; and/or (c) is not being treated for schizophrenia.

In another embodiment, the subject (a) does not have Rett Syndrome, (b) is not suspected of having or been diagnosed as having Rett Syndrome; and/or (c) is not being treated for Rett Syndrome.

In another embodiment, the subject (a) does not have autism spectrum disorder, (b) is not suspected of having or been diagnosed as having autism spectrum disorder; and/or (c) is not being treated for autism spectrum disorder.

In another embodiment, the LINE-1 inhibitor is administered to the subject as pharmaceutical composition comprising the LINE-1 inhibitor and a pharmaceutically acceptable carrier.

In another embodiment, LINE-1 inhibitor is administered to a subject as a single agent.

In another embodiment, LINE-1 inhibitor is administered to a subject in combination with one or more optional therapeutic agents, e.g., therapeutic agents that improve cognitive functioning, e.g., donepezil (Aricept®), rivastigmine tartrate (Exelon®), galantamine HBr (Reminyl®), memantine (Namenda®), or modafinil (Provigil®). In another embodiment, LINE-1 inhibitor is administered to a subject in combination with one optional therapeutic agent. In another embodiment, LINE-1 inhibitor is administered to a subject in combination with two optional therapeutic agents.

The LINE-1 inhibitor and the one or more optional therapeutic agents can be administered in combination under one or more of the following conditions: at different periodicities, at different durations, at different concentrations, by different administration routes, etc.

In another embodiment, the LINE-1 inhibitor and the one or more optional therapeutic agents are administered in combination to a subject as part of a single pharmaceutical composition.

In another embodiment, the LINE-1 inhibitor and the one or more optional therapeutic agents are administered in combination to a subject separately, e.g., as two or more separate pharmaceutical compositions. In this case, two separate pharmaceutical compositions, e.g., one comprising the LINE-1 inhibitor and one comprising an optional therapeutic agent, are administered to a subject. The separate pharmaceutical compositions can be administered to the subject, for example, at different periodicities, at different durations, by different administration routes, e.g., the LINE-1 inhibitor can be administered orally and the optional therapeutic agent(s) can be administered intravenously, or the same administration routes.

In another embodiments, the LINE-1 inhibitor is administered to the subject prior to the one or more optional therapeutic agents, e.g., 0.5, 1, 2, 3, 4, 5, 10, 12, or 18 hours, 1, 2, 3, 4, 5, or 6 days, or 1, 2, 3, or 4 weeks prior to the administration of the one or more optional therapeutic agents.

In another embodiments, the LINE-1 inhibitor is administered to the subject after the one or more optional therapeutic agents, e.g., 0.5, 1, 2, 3, 4, 5, 10, 12, or 18 hours, 1, 2, 3, 4, 5, or 6 days, or 1, 2, 3, or 4 weeks after the administration of the one or more optional therapeutic agents.

In another embodiments, the LINE-1 inhibitor and the one or more optional therapeutic agents are administered concurrently.

In another embodiment, the LINE-1 inhibitor is administered to the subject according to a continuous dosing schedule.

In another embodiment, the LINE-1 inhibitor is administered to the subject according to an intermittent dosing schedule.

In another embodiment, the LINE-1 inhibitor is orally administered to the subject.

The therapeutic methods provided herein comprise administering a LINE-1 inhibitor to a subject in an amount which is effective to achieve its intended purpose. While individual needs vary, determination of optimal ranges of effective amounts of each component is within the skill of the art. Typically, the LINE-1 inhibitor is administered in an amount from about 0.01 mg/kg to about 500 mg/kg, about 0.05 mg/kg to about 100 mg/kg, about 0.05 mg/kg to about 50 mg/kg, or about 0.05 mg/kg to about 10 mg/kg. In one embodiment, the LINE-1 inhibitor is administered once a day. In another embodiment, LINE-1 inhibitor is administered twice a day. In one embodiment, LINE-1 inhibitor is administered three times a day. In one embodiment, LINE-1 inhibitor is administered four times a day. These dosages are exemplary, but there can be individual instances in which higher or lower dosages are merited, and such are within the scope of this disclosure. In practice, the physician determines the actual dosing regimen that is most suitable for an individual subject, which can vary with the age, weight, and response of the particular subject.

The unit dose may comprise from about 0.01 mg to about 1000 mg, e.g., about 1 mg to about 500 mg, e.g., about 1 mg to about 250 mg, e.g., about 1 mg to about 150 mg, e.g., about 1 mg to about 100 mg of the LINE-1 inhibitor. For example, the unit oral dose of LINE-1 inhibitor may comprise, for example, 1 mg, 2 mg, 3 mg, 4 mg, 5 mg, 6 mg, 7 mg, 8 mg, 9 mg, or 10 mg of islatravir, or 10 mg, 25 mg, 50 mg, 75 mg, or 100 mg of censavudine. The unit dose may be administered one or more times daily, e.g., as one or more tablets or capsules. The unit dose may also be administered by any suitable route, e.g., orally, by IV, inhalation or subcutaneously to the subject. In practice, the physician determines the actual dosing regimen that is most suitable for an individual subject, which can vary with the age, weight, and response of the particular subject.

In one embodiment, the LINE-1 inhibitor is administered to a subject in an amount from about 0.1 mg to about 100 mg once a day, twice a day, three times a day, or four times a day. In another embodiment, the LINE-1 inhibitor is administered to a subject in an amount from about 1 mg to about 50 mg per day.

In one embodiment, the LINE-1 inhibitor is administered to the subject in a single dose. In another embodiment, the LINE-1 inhibitor is administered to the subject in two divided doses. In another embodiment, the LINE-1 inhibitor is administered to the subject in three divided doses. In another embodiment, the LINE-1 inhibitor is administered to the subject in four divided doses.

The LINE-1 inhibitor can be administered to a subject in the form of a raw chemical or as part of a pharmaceutical composition containing the LINE-1 inhibitor combined with a suitable pharmaceutically acceptable carrier. Such a carrier can be selected from pharmaceutically acceptable excipients, vehicles, and auxiliaries. The term “pharmaceutically acceptable carrier,” “pharmaceutically acceptable vehicle,” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, PA, 19th ed. 1995.

A pharmaceutical composition comprising the LINE-1 inhibitor can contain from about 0.01 to 99 percent by weight, e.g., from about 0.25 to 75 percent by weight, of the LINE-1 inhibitor, e.g., about 1%, about 5%, about 10%, about 15%, about 20%, about 25%, about 30%, about 35%, about 40%, about 45%, about 50%, about 55%, about 60%, about 65%, about 70%, or about 75% by weight of the LINE-1 inhibitor.

The LINE-1 inhibitor, or pharmaceutical composition comprising the LINE-1 inhibitor, can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration to a subject. Dosage forms depend on the route administration. Dosage forms include, but are not limited to, tablets, dragees, slow release lozenges, capsules, liquid solutions, liquid suspensions, oral/nasal spray, transdermal patch, thin dissolvable film, ointments, sustained or controlled release implants, mouth rinses and mouth washes, gels, hair rinses, hair gels, and shampoos, and suppositories, as well as suitable solutions for administration by intravenous infusion, and suitable suspensions for administration subcutaneous injection, and suitable powders for reconstitution. Parenteral administration can be accomplished using a needle and syringe or using other technique known in the art. In one embodiment, the LINE-1 inhibitor is administered orally to the subject. In one embodiment, the LINE-1 inhibitor is administered subcutaneously to the subject. In one embodiment, the LINE-1 inhibitor is administered intravenously to the subject.

The LINE-1 inhibitor and pharmaceutical compositions thereof may be administered to any subject who may experience the beneficial effects of LINE-1 inhibition. The term “subject” as used herein refers to any human or animal that is in need of or might benefit from therapy. Foremost among such subjects are mammals, e.g., humans, although the methods and compositions provided herein are not intended to be so limited. Other subjects include veterinary animals, e.g., cows, sheep, pigs, horses, dogs, cats and the like. In one embodiment, the subject is a human. In one embodiment, the subject is an animal.

Pharmaceutical compositions, formulations, and preparations comprising a LINE-1 inhibitor are manufactured by means of conventional mixing, granulating, dragee-making, dissolving, or lyophilizing processes. Thus, pharmaceutical preparations for oral use can be obtained by combining a LINE-1 inhibitor with solid excipients, optionally grinding the resulting mixture and processing the mixture of granules, after adding suitable auxiliaries, if desired or necessary, to obtain, e.g., tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries can be suitable flow-regulating agents and lubricants. Suitable auxiliaries include, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of LINE-1 inhibitors may be administered to a subject. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension including, for example, sodium carboxymethyl cellulose, sorbitol, and/or dextran. Optionally, the suspension may also contain stabilizers and other additives.

Therapeutically effective amounts of a LINE-1 inhibitor formulated in accordance with standard pharmaceutical practices are administered to a subject in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

Pharmaceutical compositions include those wherein a LINE-1 inhibitor is administered in an effective amount to achieve its intended purpose. The exact formulation, route of administration, and dosage is determined by an individual physician in view of the diagnosed condition or disease. Dosage amount and interval can be adjusted individually to provide levels of the LINE-1 inhibitor that is sufficient to maintain therapeutic effects.

Toxicity and therapeutic efficacy of the LINE-1 inhibitor can be determined by standard pharmaceutical procedures in cell cultures or experimental animals, e.g., for determining the maximum tolerated dose (MTD) of a compound, which defines as the highest dose that causes no toxicity in a subject. The dose ratio between the maximum tolerated dose and therapeutic effects is the therapeutic index. The dosage can vary within this range depending upon the dosage form employed, and the route of administration utilized. Determination of a therapeutically effective amount is well within the capability of those skilled in the art, especially in light of the detailed disclosure provided herein.

### II. Kits

In another embodiment, the present disclosure provides kits comprising a LINE-1 inhibitor, or a composition comprising LINE-1 inhibitor, packaged in a manner that facilitates their use to practice methods of the present disclosure.

In one embodiment, the kit includes a LINE-1 inhibitor, or a composition thereof, packaged in a container, such as a sealed bottle or vessel, with a label affixed to the container or included in the kit that describes use of the compound or composition to practice the method of the disclosure. In one embodiment, the compound or composition is packaged in a unit dosage form. The kit may include a single dose or multiple doses of a LINE-1 inhibitor, or a pharmaceutical composition thereof.

In another embodiment, the kit includes LINE-1 inhibitor, or a composition thereof, and one or more optional therapeutic agents, or a composition thereof.

### III. Definitions

The term “LINE-1 inhibitor” as used herein refers to a compound that inhibits human LINE-1 retrotransposition, e.g., with a half maximal inhibitory concentration (IC₅₀) of about 50 μM or less in a HeLa cell-based dual-luciferase assay as described in EXAMPLE 1, see below. See also Jones et al., (2008) PLoS ONE 3(2): e1547. doi:10.1371/journal.pone.0001547; Xie et al., (2011) Nucleic Acids Res. 39(3): e16. doi: 10.1093/nar/gkq1076. In another embodiment, the IC₅₀ is 1 μM or less. In another embodiment, the IC₅₀ is 0.5 μM or less. In another embodiment, the IC₅₀ is 0.25 μM or less. In another embodiment, the IC₅₀ is 0.15 μM or less. In another embodiment, the IC₅₀ is 0.1 μM or less. In another embodiment, the IC₅₀ is 0.05 μM or less. In another embodiment, the IC₅₀ is 0.01 μM or less. In another embodiment, the IC₅₀ is 0.005 μM or less. In another embodiment, the LINE-1 inhibitor is a nucleoside reverse transcriptase inhibitor (NRTI). LINE-1 inhibitors are described, for example, in WO 2020/154656. The term LINE-1 inhibitor includes, unless otherwise indicated, pharmaceutically acceptable salts and solvates of the compound that inhibit human LINE-1 retrotransposition.

In one embodiment, the LINE-1 inhibitor is islatravir, censavudine, elvucitabine, lamivudine (3TC), zidovudine (AZT), tenofovir, tenofovir disoproxil, tenofovir alafenamide, stavudine (d4T), zalcitabine (ddC), didanosine (ddI), emtricitabine (FTC), entecavir (ETV), 2′,3′-dideoxyguanosine (ddG), 2′,3′-dideoxyadenosine (ddA), 2′-fluoro-2′,3′-dideoxyarabinosyladenine (F-ddA), or abacavir (ABC).

In another embodiment, the LINE-1 inhibitor is islatravir.

In another embodiment, the LINE-1 inhibitor is censavudine.

In another embodiment, the LINE-1 inhibitor is tenofovir alafenamide.

In another embodiment, the LINE-1 inhibitor is a compound of Formula I:

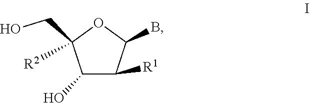

or a pharmaceutically acceptable salt or solvate thereof, or a tautomer thereof, wherein:
B is selected from the group consisting of:

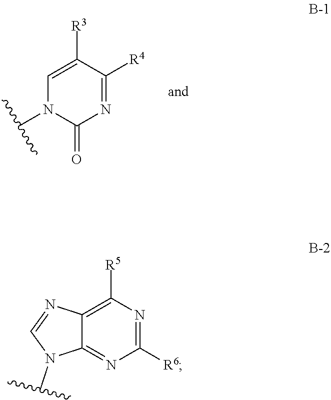

R¹ is selected from the group consisting of hydrogen and —OH;
R² is selected from the group consisting of methyl, ethynyl, and —CN;
R³ is selected from the group consisting of hydrogen, fluoro, chloro, bromo, iodo and methyl;
R⁴ is selected from the group consisting of —NH₂ and —OH;
R⁵ is selected from the group consisting of —NH₂ and —OH; and
R⁶ is selected from the group consisting of hydrogen, fluoro, chloro, and —NH₂

In another embodiment, the LINE-1 inhibitor is a compound of Formula II:

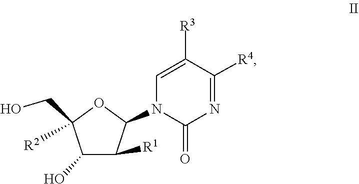

or a pharmaceutically acceptable salt or solvate thereof, or a tautomer thereof, wherein R¹, R², R³, and R⁴ are as defined in connection with Formula I.

In another embodiment, the LINE-1 inhibitor is a compound of Formula II, or a pharmaceutically acceptable salt or solvate thereof, or a tautomer thereof, wherein R³ is hydrogen.

In another embodiment, the LINE-1 inhibitor is a compound of Formula II, or a pharmaceutically acceptable salt or solvate thereof, or a tautomer thereof, wherein R³ is selected from the group consisting of fluoro and chloro.

In another embodiment, the LINE-1 inhibitor is a compound of Formula II, or a pharmaceutically acceptable salt or solvate thereof, or a tautomer thereof, wherein R³ is methyl.

In another embodiment, the LINE-1 inhibitor is a compound of Formula II, or a pharmaceutically acceptable salt or solvate thereof, or a tautomer thereof, wherein R⁴ is —NH₂.

In another embodiment, the LINE-1 inhibitor is a compound of Formula II, or a pharmaceutically acceptable salt or solvate thereof, or a tautomer thereof, wherein R⁴ is —OH.

In another embodiment, the LINE-1 inhibitor is a compound of Formula III:

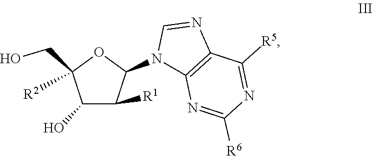

or a pharmaceutically acceptable salt or solvate thereof, or a tautomer thereof, wherein R¹, R², R⁵, and R⁶ are as defined in connection with Formula I.

In another embodiment, the LINE-1 inhibitor is a compound of Formula III, or a pharmaceutically acceptable salt or solvate thereof, or a tautomer thereof, wherein R⁵ is —NH₂.

In another embodiment, the LINE-1 inhibitor is a compound of Formula III, or a pharmaceutically acceptable salt or solvate thereof, or a tautomer thereof, wherein R⁵ is —OH.

In another embodiment, the LINE-1 inhibitor is a compound of Formula III, or a pharmaceutically acceptable salt or solvate thereof, or a tautomer thereof, wherein R⁶ is hydrogen.

In another embodiment, the LINE-1 inhibitor is a compound of Formula III, or a pharmaceutically acceptable salt or solvate thereof, or a tautomer thereof, wherein R⁶ is chloro.

In another embodiment, the LINE-1 inhibitor is a compound of Formula III, or a pharmaceutically acceptable salt or solvate thereof, or a tautomer thereof, wherein R⁶ is fluoro.

In another embodiment, the LINE-1 inhibitor is a Formula III, or a pharmaceutically acceptable salt or solvate thereof, or a tautomer thereof, wherein R⁶ is —NH₂.

In another embodiment, the LINE-1 inhibitor is a compound of any one of Formulae I-III, or a pharmaceutically acceptable salt or solvate thereof, or a tautomer thereof, wherein R¹ is hydrogen.

In another embodiment, the LINE-1 inhibitor is a compound of any one of Formulae I-III, or a pharmaceutically acceptable salt or solvate thereof, or a tautomer thereof, wherein R¹ is —OH.

In another embodiment, the LINE-1 inhibitor is a compound of any one of Formulae I-III, or a pharmaceutically acceptable salt or solvate thereof, or a tautomer thereof, wherein R² is methyl.

In another embodiment, the LINE-1 inhibitor is a compound of any one of Formulae I-III, or a pharmaceutically acceptable salt or solvate thereof, or a tautomer thereof, wherein R² is ethynyl.

In another embodiment, the LINE-1 inhibitor is a compound of any one of Formulae I-III, or a pharmaceutically acceptable salt or solvate thereof, or a tautomer thereof, wherein R² is —CN.

In another embodiment, the LINE-1 inhibitor is a compound selected from the group consisting of:

TABLE A

| Cpd. No. | Structure |
|---|---|
| 1 | 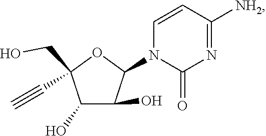 [SMILES: C#C[C@]1(CO)O[C@@H](n2ccc(N)nc2=O)[C@@H](O)[C@@H]1O] |
| 2 | 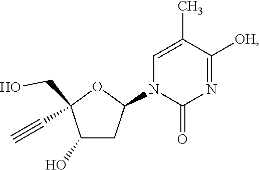 [SMILES: C#C[C@]1(CO)O[C@@H](n2cc(C)c(O)nc2=O)C[C@@H]1O] |
| 3 | 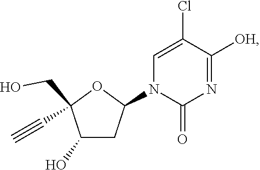 [SMILES: C#C[C@]1(CO)O[C@@H](n2cc(Cl)c(O)nc2=O)C[C@@H]1O] |
| 4 | 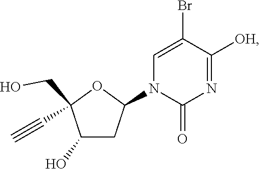 [SMILES: C#C[C@]1(CO)O[C@@H](n2cc(Br)c(O)nc2=O)C[C@@H]1O] |
| 5 | 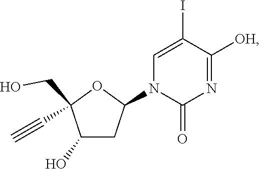 [SMILES: C#C[C@]1(CO)O[C@@H](n2cc(I)c(O)nc2=O)C[C@@H]1O] |
| 6 | 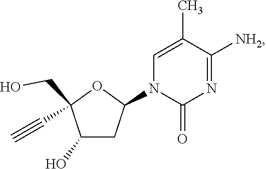 [SMILES: C#C[C@]1(CO)O[C@@H](n2cc(C)c(N)nc2=O)C[C@@H]1O] |
| 7 | 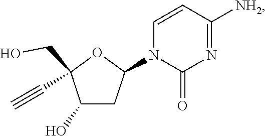 [SMILES: C#C[C@]1(CO)O[C@@H](n2ccc(N)nc2=O)C[C@@H]1O] |
| 8 | 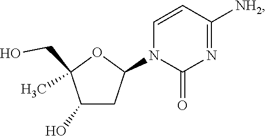 [SMILES: C[C@]1(CO)O[C@@H](n2ccc(N)nc2=O)C[C@@H]1O] |
| 9 | 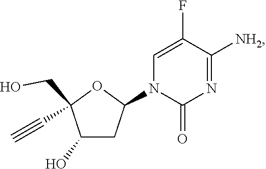 [SMILES: C#C[C@]1(CO)O[C@@H](n2cc(F)c(N)nc2=O)C[C@@H]1O] |
| 10 | 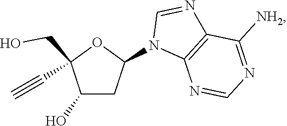 [SMILES: C#C[C@]1(CO)O[C@@H](n2cnc3c(N)ncnc32)C[C@@H]1O] |
| 11 | 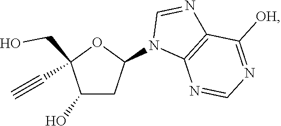 [SMILES: C#C[C@]1(CO)O[C@@H](n2cnc3c(O)ncnc32)C[C@@H]1O] |
| 12 | 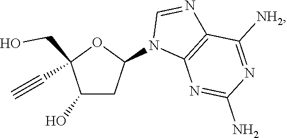 [SMILES: C#C[C@]1(CO)O[C@@H](n2cnc3c(N)nc(N)nc32)C[C@@H]1O] |
| 13 | 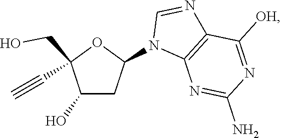 [SMILES: C#C[C@]1(CO)O[C@@H](n2cnc3c(O)nc(N)nc32)C[C@@H]1O] |
| 14 | 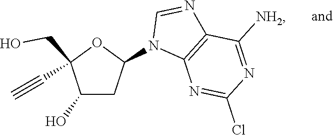 [SMILES: C#C[C@]1(CO)O[C@@H](n2cnc3c(N)nc(Cl)nc32)C[C@@H]1O] |
| 15 | 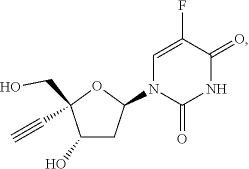 [SMILES: C#C[C@]1(CO)O[C@@H](n2cc(F)c(=O)[nH]c2=O)C[C@@H]1O] |

or a pharmaceutically acceptable salt or solvate thereof, or a tautomer thereof.

In another embodiment, the LINE-1 inhibitor is a compound selected from the group consisting of:

TABLE B

| Cpd. No. | Structure |
|---|---|
| 16 | 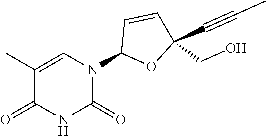 [SMILES: CC#C[C@]1(CO)C=C[C@H](n2cc(C)c(=O)[nH]c2=O)O1] |
| 17 | 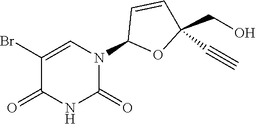 [SMILES: C#C[C@@]1(CO)C=C[C@H](n2cc(Br)c(=O)[nH]c2=O)O1] |
| 18 | 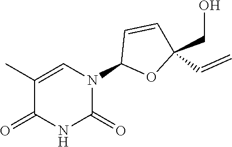 [SMILES: C=C[C@@]1(CO)C=C[C@H](n2cc(C)c(=O)[nH]c2=O)O1] |
| 19 | 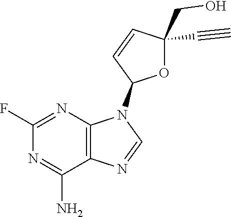 [SMILES: C#C[C@@]1(CO)C=C[C@H](n2cnc3c(N)nc(F)nc32)O1] |
| 20 | 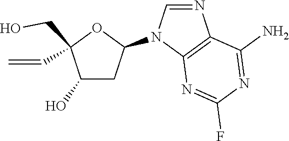 [SMILES: C=C[C@]1(CO)O[C@@H](n2cnc3c(N)nc(F)nc32)C[C@@H]1O] |
| 21 | 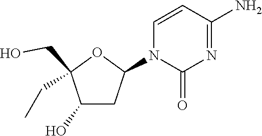 [SMILES: CC[C@]1(CO)O[C@@H](n2ccc(N)nc2=O)C[C@@H]1O] |
| 22 | 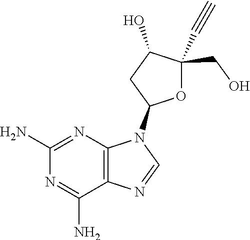 [SMILES: C#C[C@]1(CO)O[C@@H](n2cnc3c(N)nc(N)nc32)C[C@@H]1O] |
| 23 | 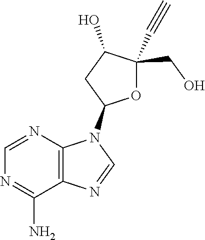 [SMILES: C#C[C@]1(CO)O[C@@H](n2cnc3c(N)ncnc32)C[C@@H]1O] |
| 24 | 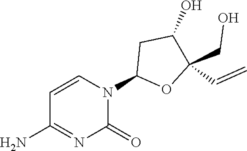 [SMILES: C=C[C@]1(CO)O[C@@H](n2ccc(N)nc2=O)C[C@@H]1O] |
| 25 | 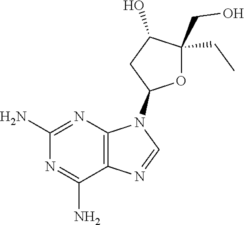 [SMILES: CC[C@]1(CO)O[C@@H](n2cnc3c(N)nc(N)nc32)C[C@@H]1O] |
| 26 | 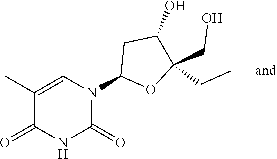 [SMILES: CC[C@]1(CO)O[C@@H](n2cc(C)c(=O)[nH]c2=O)C[C@@H]1O] |
| 27 | 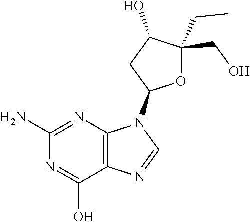 [SMILES: CC[C@]1(CO)O[C@@H](n2cnc3c(O)nc(N)nc32)C[C@@H]1O] |

or a pharmaceutically acceptable salt or solvate thereof, or a tautomer thereof.

In another embodiment, the LINE-1 inhibitor is a compound selected from the group consisting of:

TABLE C

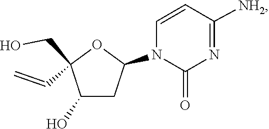 [SMILES: C=C[C@]1(CO)O[C@@H](n2ccc(N)nc2=O)C[C@@H]1O]

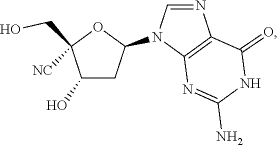 [SMILES: N#C[C@]1(CO)O[C@@H](n2cnc3c(=O)[nH]c(N)nc32)C[C@@H]1O]

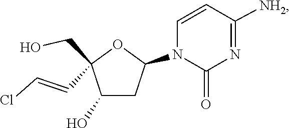 [SMILES: Nc1ccn([C@H]2C[C@H](O)[C@@](/C=C/Cl)(CO)O2)c(=O)n1]

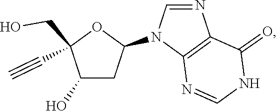 [SMILES: C#C[C@]1(CO)O[C@@H](n2cnc3c(=O)[nH]cnc32)C[C@@H]1O]

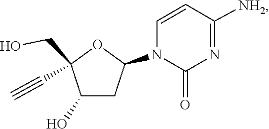 [SMILES: C#C[C@]1(CO)O[C@@H](n2ccc(N)nc2=O)C[C@@H]1O]

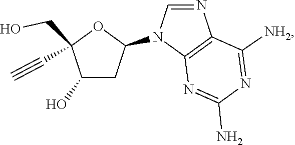 [SMILES: C#C[C@]1(CO)O[C@@H](n2cnc3c(N)nc(N)nc32)C[C@@H]1O]

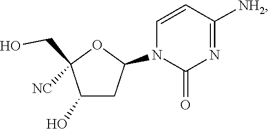 [SMILES: N#C[C@]1(CO)O[C@@H](n2ccc(N)nc2=O)C[C@@H]1O]

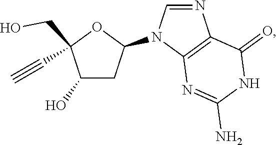 [SMILES: C#C[C@]1(CO)O[C@@H](n2cnc3c(=O)[nH]c(N)nc32)C[C@@H]1O]

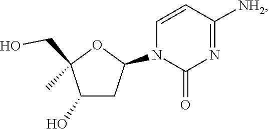 [SMILES: C[C@]1(CO)O[C@@H](n2ccc(N)nc2=O)C[C@@H]1O]

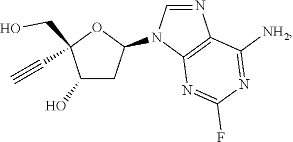 [SMILES: C#C[C@]1(CO)O[C@@H](n2cnc3c(N)nc(F)nc32)C[C@@H]1O]

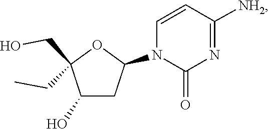 [SMILES: CC[C@]1(CO)O[C@@H](n2ccc(N)nc2=O)C[C@@H]1O]

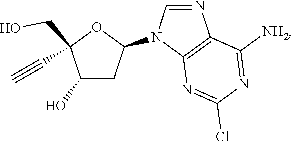 [SMILES: C#C[C@]1(CO)O[C@@H](n2cnc3c(N)nc(Cl)nc32)C[C@@H]1O]

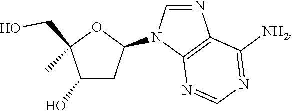 [SMILES: C[C@]1(CO)O[C@@H](n2cnc3c(N)ncnc32)C[C@@H]1O]

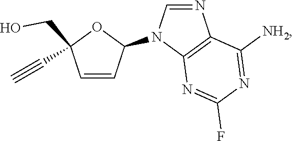 [SMILES: C#C[C@@]1(CO)C=C[C@H](n2cnc3c(N)nc(F)nc32)O1]

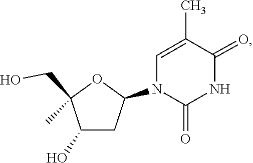 [SMILES: Cc1cn([C@H]2C[C@H](O)[C@@](C)(CO)O2)c(=O)[nH]c1=O]

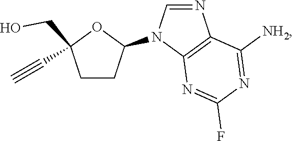 [SMILES: C#C[C@@]1(CO)CC[C@H](n2cnc3c(N)nc(F)nc32)O1]

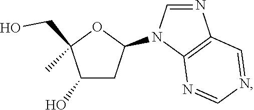 [SMILES: C[C@]1(CO)O[C@@H](n2cnc3cncnc32)C[C@@H]1O]

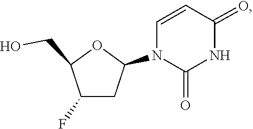 [SMILES: O=c1ccn([C@H]2C[C@H](F)[C@@H](CO)O2)c(=O)[nH]1]

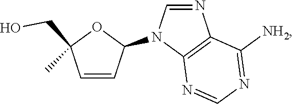 [SMILES: C[C@@]1(CO)C=C[C@H](n2cnc3c(N)ncnc32)O1]

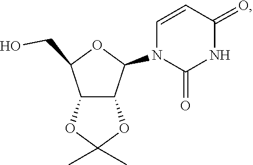 [SMILES: CC1(C)O[C@@H]2[C@H](O1)[C@@H](CO)O[C@H]2n1ccc(=O)[nH]c1=O]

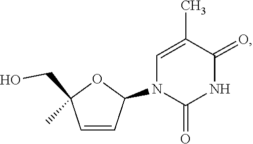 [SMILES: Cc1cn([C@H]2C=C[C@@](C)(CO)O2)c(=O)[nH]c1=O]

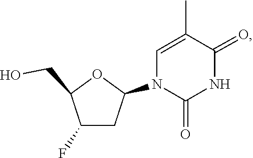 [SMILES: Cc1cn([C@H]2C[C@H](F)[C@@H](CO)O2)c(=O)[nH]c1=O]

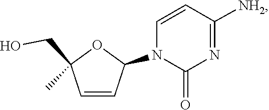 [SMILES: C[C@@]1(CO)C=C[C@H](n2ccc(N)nc2=O)O1]

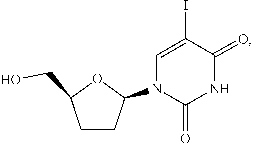 [SMILES: O=c1[nH]c(=O)n([C@H]2CC[C@@H](CO)O2)cc1I]

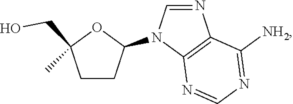 [SMILES: C[C@@]1(CO)CC[C@H](n2cnc3c(N)ncnc32)O1]

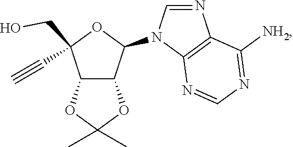 [SMILES: C#C[C@]1(CO)O[C@@H](n2cnc3c(N)ncnc32)[C@@H]2OC(C)(C)O[C@@H]21]

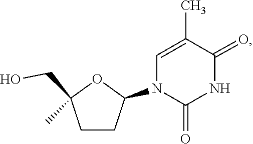 [SMILES: Cc1cn([C@H]2CC[C@@](C)(CO)O2)c(=O)[nH]c1=O]

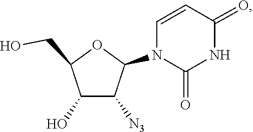 [SMILES: [N-]=[N+]=N[C@@H]1[C@H](O)[C@@H](CO)O[C@H]1n1ccc(=O)[nH]c1=O]

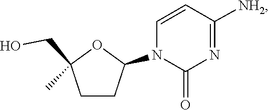 [SMILES: C[C@@]1(CO)CC[C@H](n2ccc(N)nc2=O)O1]

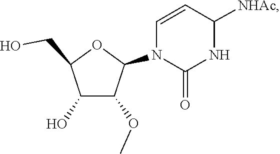 [SMILES: CO[C@@H]1[C@H](O)[C@@H](CO)O[C@H]1N1C=CC(NC(C)=O)NC1=O]

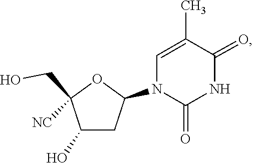 [SMILES: Cc1cn([C@H]2C[C@H](O)[C@@](C#N)(CO)O2)c(=O)[nH]c1=O]

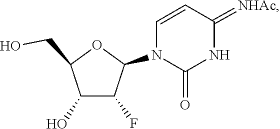

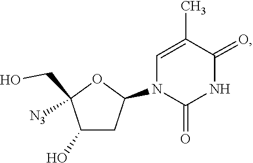 [SMILES: Cc1cn([C@H]2C[C@H](O)[C@](CO)(N=[N+]=[N-])O2)c(=O)[nH]c1=O]

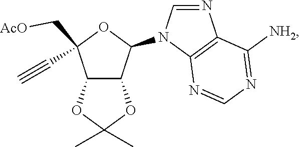 [SMILES: C#C[C@]1(COC(C)=O)O[C@@H](n2cnc3c(N)ncnc32)[C@@H]2OC(C)(C)O[C@@H]21]

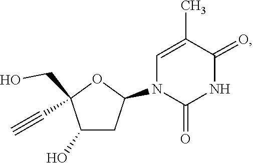 [SMILES: C#C[C@]1(CO)O[C@@H](n2cc(C)c(=O)[nH]c2=O)C[C@@H]1O]

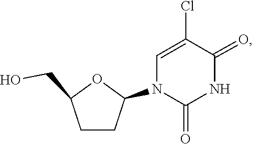 [SMILES: O=c1[nH]c(=O)n([C@H]2CC[C@@H](CO)O2)cc1Cl]

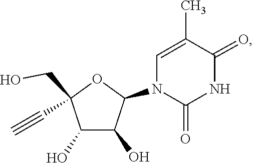 [SMILES: C#C[C@]1(CO)O[C@@H](n2cc(C)c(=O)[nH]c2=O)[C@@H](O)[C@@H]1O]

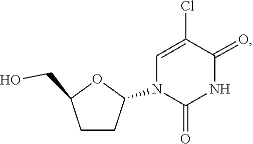 [SMILES: O=c1[nH]c(=O)n([C@@H]2CC[C@@H](CO)O2)cc1Cl]

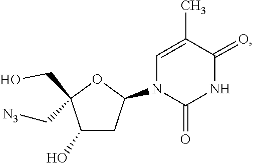 [SMILES: Cc1cn([C@H]2C[C@H](O)[C@](CO)(CN=[N+]=[N-])O2)c(=O)[nH]c1=O]

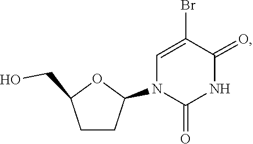 [SMILES: O=c1[nH]c(=O)n([C@H]2CC[C@@H](CO)O2)cc1Br]

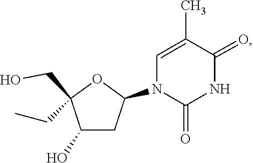 [SMILES: CC[C@]1(CO)O[C@@H](n2cc(C)c(=O)[nH]c2=O)C[C@@H]1O]

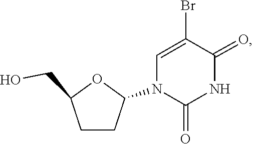 [SMILES: O=c1[nH]c(=O)n([C@@H]2CC[C@@H](CO)O2)cc1Br]

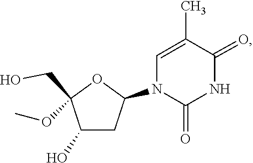 [SMILES: CO[C@]1(CO)O[C@@H](n2cc(C)c(=O)[nH]c2=O)C[C@@H]1O]

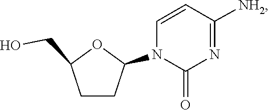 [SMILES: Nc1ccn([C@H]2CC[C@@H](CO)O2)c(=O)n1]

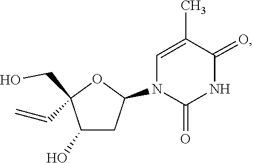 [SMILES: C=C[C@]1(CO)O[C@@H](n2cc(C)c(=O)[nH]c2=O)C[C@@H]1O]

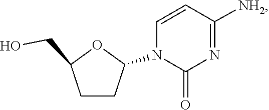 [SMILES: Nc1ccn([C@@H]2CC[C@@H](CO)O2)c(=O)n1]

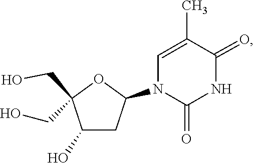

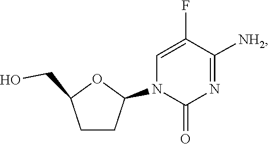 [SMILES: Nc1nc(=O)n([C@H]2CC[C@@H](CO)O2)cc1F]

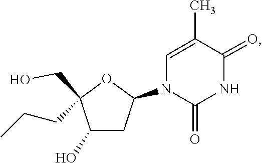 [SMILES: CCC[C@]1(CO)O[C@@H](n2cc(C)c(=O)[nH]c2=O)C[C@@H]1O]

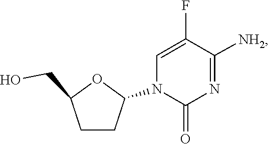 [SMILES: Nc1nc(=O)n([C@@H]2CC[C@@H](CO)O2)cc1F]

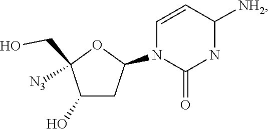 [SMILES: [N-]=[N+]=N[C@]1(CO)O[C@@H](N2C=CC(N)NC2=O)C[C@@H]1O]

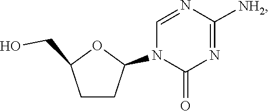 [SMILES: Nc1ncn([C@H]2CC[C@@H](CO)O2)c(=O)n1]

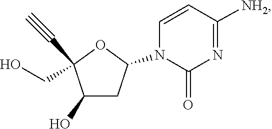 [SMILES: C#C[C@@]1(CO)O[C@H](n2ccc(N)nc2=O)C[C@H]1O]

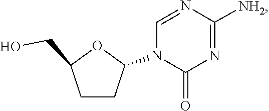 [SMILES: Nc1ncn([C@@H]2CC[C@@H](CO)O2)c(=O)n1]

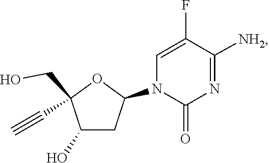 [SMILES: C#C[C@]1(CO)O[C@@H](n2cc(F)c(N)nc2=O)C[C@@H]1O]

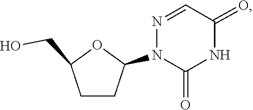 [SMILES: O=c1cnn([C@H]2CC[C@@H](CO)O2)c(=O)[nH]1]

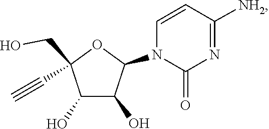 [SMILES: C#C[C@]1(CO)O[C@@H](n2ccc(N)nc2=O)[C@@H](O)[C@@H]1O]

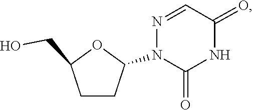 [SMILES: O=c1cnn([C@@H]2CC[C@@H](CO)O2)c(=O)[nH]1]

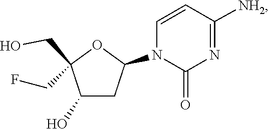 [SMILES: Nc1ccn([C@H]2C[C@H](O)[C@](CO)(CF)O2)c(=O)n1]

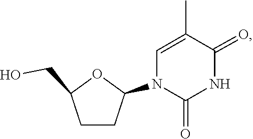 [SMILES: Cc1cn([C@H]2CC[C@@H](CO)O2)c(=O)[nH]c1=O]

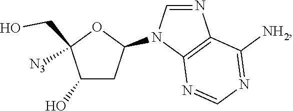 [SMILES: [N-]=[N+]=N[C@]1(CO)O[C@@H](n2cnc3c(N)ncnc32)C[C@@H]1O]

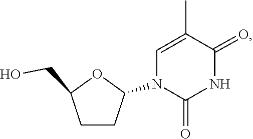 [SMILES: Cc1cn([C@@H]2CC[C@@H](CO)O2)c(=O)[nH]c1=O]

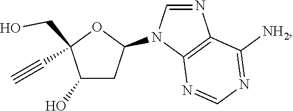 [SMILES: C#C[C@]1(CO)O[C@@H](n2cnc3c(N)ncnc32)C[C@@H]1O]

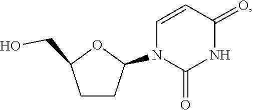 [SMILES: O=c1ccn([C@H]2CC[C@@H](CO)O2)c(=O)[nH]1]

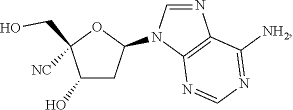 [SMILES: N#C[C@]1(CO)O[C@@H](n2cnc3c(N)ncnc32)C[C@@H]1O]

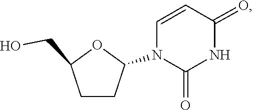 [SMILES: O=c1ccn([C@@H]2CC[C@@H](CO)O2)c(=O)[nH]1]

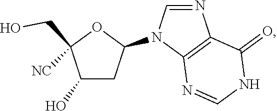 [SMILES: N#C[C@]1(CO)O[C@@H](n2cnc3c(=O)[nH]cnc32)C[C@@H]1O]

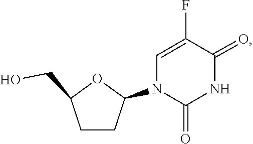 [SMILES: O=c1[nH]c(=O)n([C@H]2CC[C@@H](CO)O2)cc1F]

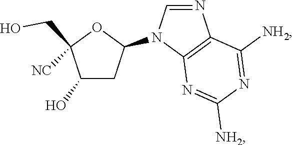 [SMILES: N#C[C@]1(CO)O[C@@H](n2cnc3c(N)nc(N)nc32)C[C@@H]1O]

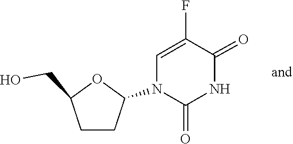 [SMILES: O=c1[nH]c(=O)n([C@@H]2CC[C@@H](CO)O2)cc1F]

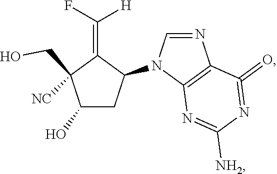

or a pharmaceutically acceptable salt or solvate thereof, or a tautomer thereof.

The compounds of Tables A-C may be found, for example, in Nomura et al., J. Med. Chem. 42:2901-2908 (1999); Ohrui, H., Proc. Jpn. Acad. Ser. B 87:53-65 (2011); Banuelos-Sanchez et al., Cell Chemical Biology 26:1095-1109 (2019); Kirby et al., Antimicrobial Agents and Chemotherapy 57:6254-6264 (2013), or JP Patent No. 6767011.

The term “tautomer” as used herein refers to each of two or more isomers of a compound which exist together in equilibrium, and are interchanged by migration of an atom, e.g., a hydrogen, or group within the molecule. Certain LINE-1 inhibitors may exist as tautomers. In situations where tautomers are possible, the present disclosure includes all tautomeric forms. For example, as illustrated in Chart 1, both the lactim and lactam tautomers are encompassed by Formula II when R⁴ is —OH, and both the amino and imino tautomers are encompassed by Formula II when R⁴ is —NH₂.

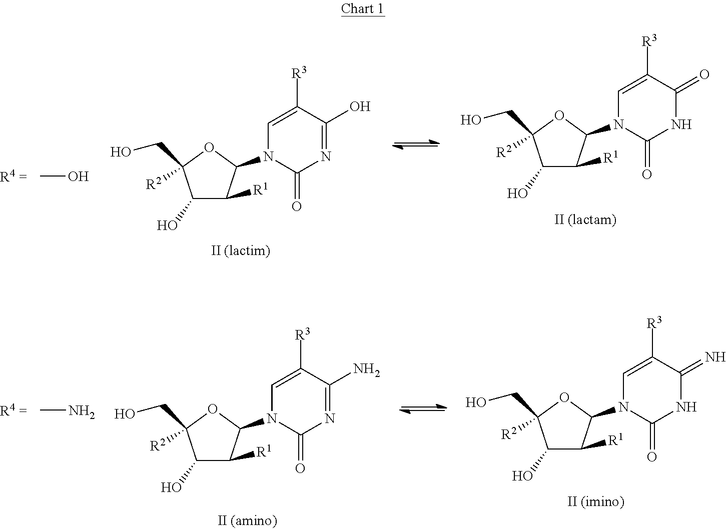

Likewise, as illustrated in Chart 2, both the lactim and lactam tautomers are encompassed by Formula III when R⁵ is —OH, and both the amino and imino tautomers are encompassed by Formula III when R⁵ is —NH₂.

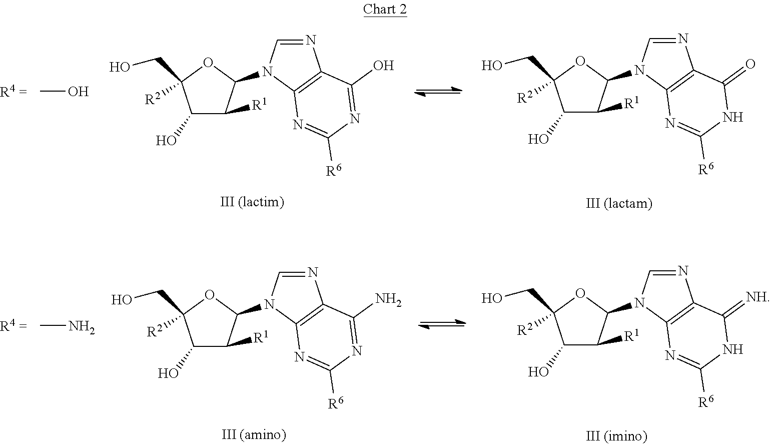

The equilibrium arrows in Charts 1 and 2 are not intended to show the position of the equilibrium, only that an equilibrium exists between the two tautomeric forms.

In one embodiment, the LINE-1 inhibitor is islatravir. Islatravir is a compound having the following chemical structure:

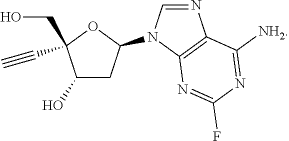 [SMILES: C#C[C@]1(CO)O[C@@H](n2cnc3c(N)nc(F)nc32)C[C@@H]1O]

Islatravir (also known as EDdA, MK-8591 or 2′-deoxy-4′-ethynyl-2-fluoroadenosine) and its method of synthesis is described in U.S. Pat. No. 7,625,877.

In some embodiments, islatravir is administered to a subject daily in an amount that ranges from about 0.1 mg to about 20 mg, e.g., from about 0.5 mg to about 15 mg, e.g., from about 1 mg to about 10 mg. In some embodiments, about 0.1 mg, about 0.15 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 1 mg, about 2 mg, about 3 mg, about 4 mg, about 5 mg, about 6 mg, about 7 mg, about 8 mg, about 9 mg, or about 10 mg of islatravir is administered to the subject per day.

In one embodiment, the LINE-1 inhibitor is censavudine. Censavudine is a compound having the following chemical structure:

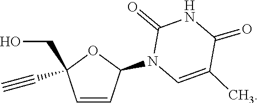 [SMILES: C#C[C@@]1(CO)C=C[C@H](n2cc(C)c(=O)[nH]c2=O)O1]

Censavudine (also known as 4′-Ed4T, 4′-ethynyl-d4T, 4′-ethynylstavudine, BMS-986001, OBP-601, festinavir) and its method of synthesis is described in U.S. Pat. No. 7,589,078.

In one embodiment, the LINE-1 inhibitor is elvucitabine. Elvucitabine is a compound having the following chemical structure:

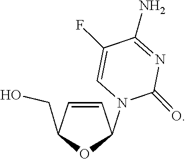 [SMILES: Nc1nc(=O)n([C@@H]2C=C[C@H](CO)O2)cc1F]

Elvucitabine and its method of synthesis is described in U.S. Pat. No. 5,627,160.

The terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language, e.g., “such as,” provided herein, is intended to better illustrate the disclosure and is not a limitation on the scope of the disclosure unless otherwise claimed. No language in the specification should be construed as indicating any non-claimed element as essential to the practice of the disclosure.

The term “about,” as used herein, includes the recited number±10%. Thus, “about 10” means 9 to 11.

As used herein, the terms “enhancing cognition” or “cognitive enhancement” and the like refer to increasing or improving the level of at least one aspect of cognitive performance, e.g., over a baseline level prior to treatment according to a method as provided herein. For example, according to some embodiments, cognitive enhancement is achieved in a subject having a cognitive deficit that is stable, i.e., not in continuing decline. According to other embodiments, the subject has a cognitive deficit that is ameliorating with time, for example during natural or medically assisted recovery from traumatic, tumor-related or ischemic brain injury. In such a subject, a method of the present disclosure can provide cognitive enhancement to a greater degree or in a shorter period of time than would occur otherwise. Cognitive enhancement can be, but is not necessarily, assessed by comparison with placebo treatment.

As used herein, the terms “inhibiting cognitive decline” and the like refer to any of slowing, retarding, delaying, reducing, arresting, and reversing progress of decline in the level of at least one aspect of cognitive performance. In other words, cognitive decline inhibition is marked by the subject exhibiting a higher level of at least one aspect of cognitive performance than the subject would have exhibited in absence of treatment according to a method as provided herein, but not necessarily a higher level than at baseline. Cognitive decline inhibition can be, but is not necessarily, assessed by comparison with placebo treatment.

As used herein, the terms “treat,” “treating,” “treatment,” and the like refer to eliminating, reducing, or ameliorating a cognitive deficit disorder and/or symptoms associated therewith. Although not precluded, treating the cognitive deficit does not require that the cognitive deficit or symptoms associated therewith be completely eliminated. However, in one embodiment, administration of a LINE-1 inhibitor leads to elimination of the cognitive and associated symptoms.

As used herein, the terms “prevent,” “preventing,” “prevention” and the like refer to a method of preventing the onset of a cognitive deficit disorder and/or symptoms associated therewith, or barring a subject from acquiring a cognitive deficit disorder. The terms “prevent,” “preventing,” and “prevention” also include delaying the onset of a cognitive deficit disorder and/or its attendant symptoms, and reducing a subject's risk of acquiring the cognitive deficit disorder. The terms “prevent,” “preventing” and “prevention” includes “prophylactic treatment,” which refers to reducing the probability of redeveloping a cognitive deficit disorder, or of a recurrence of a previously-controlled cognitive deficit disorder in a subject who does not have, but is at risk of or is susceptible to, redeveloping the cognitive deficit disorder or a recurrence of the cognitive deficit disorder.

Aspects of cognitive performance which can be improved, or decline in which can be slowed, retarded, delayed, reduced, arrested or reversed include, without limitation, memory acquisition, memory retention, sensory perception, learning, verbal and numerical skills, social skills, and/or communication skills. A beneficial effect on at least one cognitive performance aspect can represent successful treatment, but in some cases more than one aspect of cognitive performance exhibits a beneficial response. One or more cognitive tests can be used to check for cognitive issues including, but not limited to, the Montreal Cognitive Assessment (MoCA) test, the General Practitioner assessment of Cognition (GPCOG), the Mini-Mental State Exam (MMSE), and the Mini-Cog test.

As used herein, the term “cognitive deficit disorder” refers to any disorder in which the subject exhibits an abnormally low level of at least one aspect of cognitive performance. Cognitive deficit disorders treatable by methods provided herein include without limitation learning disorders, memory disorders, sensory perception disorders, attention deficit/hyperactivity disorder, cognitive deficits associated with autism or Asperger's syndrome, mild cognitive impairment, age-related cognitive decline, cognitive impairment associated with traumatic, tumor-related or ischemic brain injury (including acute cerebrovascular events such as stroke, hemorrhage, embolism, thrombosis or rupturing aneurysm), drug- or alcohol-related cognitive impairment, and the like.

The term “therapeutically effective amount,” as used herein, refers to that amount of a LINE-1 inhibitor sufficient to result, e.g., in the amelioration of one or more symptoms of a cognitive deficit disorder, or prevent advancement of a cognitive deficit, or cause regression of a cognitive deficit disorder. For example, with respect to the treatment of cognitive deficit disorder, in one embodiment, a therapeutically effective amount will refer to the amount of a LINE-1 inhibitor that causes a beneficial effect on at least one aspect of cognitive performance. The therapeutically effective amount is typically determined by the attendant physician. For example, dosage amounts and intervals can be adjusted individually to provide plasma levels of a LINE-1 inhibitor that are sufficient to maintain the desired therapeutic effects. The desired dose conveniently can be administered in a single dose, or as multiple doses administered at appropriate intervals, for example as one, two, three, four or more subdoses per day.

The term “container” means any receptacle and closure therefore suitable for storing, shipping, dispensing, and/or handling the LINE-1 inhibitor. Non-limiting exemplary containers include vials, ampules, bottles, and syringes.

The term “insert” means information accompanying a pharmaceutical product that provides a description of how to administer the product, along with the safety and efficacy data required to allow the physician, pharmacist, and subject to make an informed decision regarding use of the product. The package insert generally is regarded as the “label” for a pharmaceutical product.

In some embodiments, when administered in combination, two or more therapeutic agents can have a synergistic effect. The terms “synergy,” “synergistic,” “synergistically” and derivations thereof, such as in a “synergistic effect” or a “synergistic combination” or a “synergistic composition” as used herein refer to circumstances under which the biological activity of a combination of an agent and at least one additional therapeutic agent is greater than the sum of the biological activities of the respective agents when administered individually. For example, the term “synergistically effective” as used herein refers to the interaction between a LINE-1 inhibitor and another therapeutic agent that causes the total effect of the drugs to be greater than the sum of the individual effects of each drug. Berenbaum, Pharmacological Reviews 41:93-141 (1989).

The terms “intermittent dose administration,” “intermittent dosing schedule,” and similar terms as used herein refer to, i.e., not continuous, administration, of a LINE-1 inhibitor to a subject.

Intermittent dose administration of a LINE-1 inhibitor may maintain or improve the efficacy achieved with continuous dosing, but with less side-effects, e.g., less body weight loss. Intermittent dose administration regimens useful in the present disclosure encompass any discontinuous administration regimen that provides a therapeutically effective amount of a LINE-1 inhibitor to a subject in need thereof. Intermittent dosing regimens can use equivalent, lower, or higher doses of the LINE-1 inhibitor than would be used in continuous dosing regimens. Advantages of intermittent dose administration of a LINE-1 inhibitor include, but are not limited to, improved safety, decreased toxicity, e.g., decreased weight loss, increased exposure, increased efficacy, and/or increased subject compliance. These advantages may be realized when the LINE-1 inhibitor is administered as a single agent or when administered in combination with one or more optional therapeutic agents. On the day a LINE-1 inhibitor is scheduled to be administered to the subject, administration can occur in a single or in divided doses, e.g., once-a-day, twice-a-day, three times a day, four times a day or more. Dosing can also occur via any suitable route, e.g., orally, intravenously, or subcutaneously. In one embodiment, the LINE-1 inhibitor is administered to the subject once (QD) or twice (BID) on the day the compound is scheduled to be administered.

The phrase “in combination” as used in connection with the administration of a LINE-1 inhibitor and one or more optional therapeutic agents to a subject means that the LINE-1 inhibitor and the one or more optional therapeutic agents can be administered to the subject together, e.g., as part of a single pharmaceutical composition or formulation, or separately, e.g., as part of two or more separate pharmaceutical compositions or formulations. The phrase “in combination” as used in connection with the administration of a LINE-1 inhibitor and the one or more optional therapeutic agents to a subject is thus intended to embrace administration of the LINE-1 inhibitor and the one or more optional therapeutic agents in a sequential manner, wherein the LINE-1 inhibitor and the one or more optional therapeutic agents are administered to the subject at a different time, as well as administration concurrently, or in a substantially simultaneous manner, e.g., less than 30 minutes apart. Simultaneous administration can be accomplished, for example, by administering to the subject a single capsule having a fixed ratio of each of the LINE-1 inhibitor and the one or more optional therapeutic agents or in multiple, single capsules for each of the LINE-1 inhibitor and the one or more optional therapeutic agents. Sequential or substantially simultaneous administration of the LINE-1 inhibitor and the one or more optional therapeutic agents can be accomplished by any appropriate route including, but not limited to, oral routes, intravenous routes, subcutaneous routes, intramuscular routes, etc. The LINE-1 inhibitor and the one or more optional therapeutic agents can be administered by the same route or by different routes. For example, the one or more optional therapeutic agents and the LINE-1 inhibitor of the combination may be administered orally. Alternatively, for example, the LINE-1 inhibitor may be administered orally and the one or more optional therapeutic agents may be administered by intravenous injection. The LINE-1 inhibitor and the one or more optional therapeutic agents may also be administered in alternation. In one embodiment, the LINE-1 inhibitor and the one or more optional therapeutic agents are administered to a subject separately, e.g., as part of two or more separate pharmaceutical compositions or formulations.

### IV. Particular Embodiments

The disclosure provides the following particular embodiments.

Embodiment 1. A method to enhance cognition, inhibit cognitive decline, treat or prevent a cognitive deficit disorder, or treat or prevent CJD in a subject in need thereof, the method comprising administering to the subject a therapeutically effective amount of a LINE-1 inhibitor.

Embodiment 2. The method of Embodiment 1, wherein the LINE-1 inhibitor is administered to enhance cognition.

Embodiment 3. The method of Embodiment 1, wherein the LINE-1 inhibitor is administered to inhibit cognitive decline.

Embodiment 4. The method of Embodiment 1, wherein the LINE-1 inhibitor is administered to treat or prevent a cognitive deficit disorder.

Embodiment 5. The method of Embodiment 4, wherein the cognitive deficit disorder is a learning disorder, a memory disorder, a sensory perception disorder, an attention deficit/hyperactivity disorder, associated with autism or Asperger's syndrome, mild cognitive impairment, age-related cognitive decline, associated with traumatic, tumor-related, or ischemic brain injury, a drug-related cognitive impairment, or an alcohol-related cognitive impairment.

Embodiment 6. The method of any one of Embodiments 1-5, wherein the LINE-1 inhibitor is islatravir, censavudine, elvucitabine, lamivudine (3TC), zidovudine (AZT), tenofovir, tenofovir disoproxil, tenofovir alafenamide, stavudine (d4T), zalcitabine (ddC), didanosine (ddI), emtricitabine (FTC), entecavir (ETV), 2′,3′-dideoxyguanosine (ddG), 2′,3′-dideoxyadenosine (ddA), 2′-fluoro-2′,3′-dideoxyarabinosyladenine (F-ddA), or abacavir (ABC).

Embodiment 7. The method of Embodiment 6, wherein the LINE-1 inhibitor is islatravir.

Embodiment 8. The method of Embodiment 6, wherein the LINE-1 inhibitor is censavudine.

Embodiment 9. The method of any one of Embodiments 1-5, wherein the LINE-1 inhibitor is a compound of Formula I:

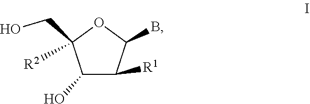

or a pharmaceutically acceptable salt or solvate thereof, or a tautomer thereof, wherein:
B is selected from the group consisting of:

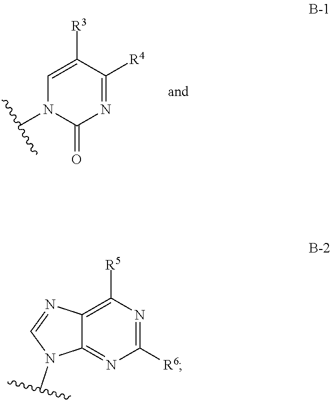

R¹ is selected from the group consisting of hydrogen and —OH;
R² is selected from the group consisting of methyl, ethynyl, and —CN;
R³ is selected from the group consisting of hydrogen, fluoro, chloro, bromo, iodo and methyl;
R⁴ is selected from the group consisting of —NH₂ and —OH;
R⁵ is selected from the group consisting of —NH₂ and —OH; and
R⁶ is selected from the group consisting of hydrogen, fluoro, chloro, and —NH₂.

Embodiment 10. The method of Embodiment 9, wherein the LINE-1 inhibitor is a compound of Formula II:

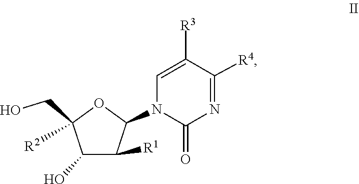

or a pharmaceutically acceptable salt or solvate thereof, or a tautomer thereof.

Embodiment 11. The method of Embodiment 10, wherein R³ is hydrogen.

Embodiment 12. The method of Embodiment 10, wherein R³ is selected from the group consisting of fluoro and chloro.

Embodiment 13. The method of Embodiment 10, wherein R³ is methyl.

Embodiment 14. The method of any one of Embodiments 10-13, wherein R⁴ is —NH₂.

Embodiment 15. The method of any one of Embodiments 10-13, wherein R⁴ is —OH.

Embodiment 16. The method of Embodiment 9, wherein the LINE-1 inhibitor is a compound of Formula III:

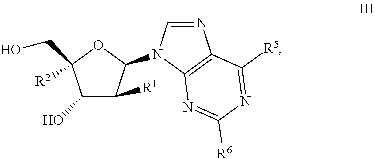

or a pharmaceutically acceptable salt or solvate thereof, or a tautomer thereof.

Embodiment 17. The method of Embodiment 16, wherein R⁵ is —NH₂.

Embodiment 18. The method of Embodiment 16, wherein R⁵ is —OH.

Embodiment 19. The method of any one of Embodiments 16-18, wherein R⁶ is hydrogen.

Embodiment 20. The method of any one of Embodiments 16-18, wherein R⁶ is chloro.

Embodiment 21. The method of any one of Embodiments 16-18, wherein R⁶ is fluoro.

Embodiment 22. The method of any one of Embodiments 16-28, wherein R⁶ is —NH₂.

Embodiment 23. The method of any one of Embodiments 9-22, wherein R¹ is hydrogen.

Embodiment 24. The method of any one of Embodiments 9-22, wherein R¹ is —OH.

Embodiment 25. The method of any one of Embodiments 9-24, wherein R² is methyl.

Embodiment 26. The method of any one of Embodiments 9-24, wherein R² is ethynyl.

Embodiment 27. The method of any one of Embodiments 9-24, wherein R² is —CN.

Embodiment 28. The method of Embodiment 9, wherein the LINE-1 inhibitor is a compound selected from the group consisting of:

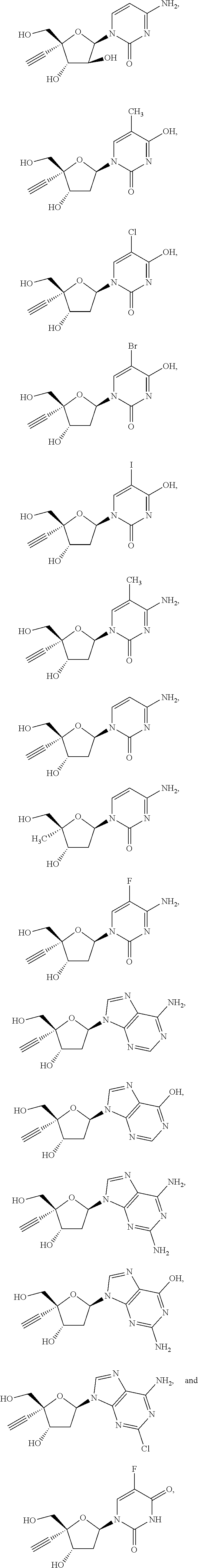

or a pharmaceutically acceptable salt or solvate thereof, or a tautomer thereof, or a compound of Table B, or a pharmaceutically acceptable salt or solvate thereof, or a tautomer thereof, or a compound of Table C, or a pharmaceutically acceptable salt or solvate thereof, or a tautomer thereof.

Embodiment 29. The method of any one of Embodiments 1-28, wherein the LINE-1 inhibitor is administered as a pharmaceutical composition comprising the LINE-1 inhibitor and pharmaceutically acceptable carrier.

Embodiment 30. The method of any one of Embodiments 1-29 further comprising administering one or more optional therapeutic agents to the subject.

Embodiment 31. The method of any one of Embodiments 1-30, wherein the subject is (a) not infected with the HIV virus, (b) is not suspected of being infected with the HIV virus, and/or (c) is not being treated to prevent infection with the HIV virus.

Embodiment 32. A LINE-1 inhibitor for use to enhance cognition, inhibit cognitive decline, treat or prevent a cognitive deficit disorder, or treat or prevent CJD in a subject in need thereof.

Embodiment 33. The LINE-1 inhibitor for use of Embodiment 32, wherein the LINE-1 inhibitor is administered to enhance cognition.

Embodiment 34. The LINE-1 inhibitor for use of Embodiment 32, wherein the LINE-1 inhibitor is administered to inhibit cognitive decline.

Embodiment 35. The LINE-1 inhibitor for use of Embodiment 32, wherein the LINE-1 inhibitor is administered to treat or prevent a cognitive deficit disorder.

Embodiment 36. The LINE-1 inhibitor for use of Embodiment 35, wherein the cognitive deficit disorder is a learning disorder, a memory disorder, a sensory perception disorder, an attention deficit/hyperactivity disorder, associated with autism or Asperger's syndrome, mild cognitive impairment, age-related cognitive decline, associated with traumatic, tumor-related, or ischemic brain injury, a drug-related cognitive impairment, or an alcohol-related cognitive impairment.

Embodiment 37. The LINE-1 inhibitor for use of any one of Embodiments 32-36, wherein the LINE-1 inhibitor is islatravir, censavudine, elvucitabine, lamivudine (3TC), zidovudine (AZT), tenofovir, tenofovir disoproxil, tenofovir alafenamide, stavudine (d4T), zalcitabine (ddC), didanosine (ddI), emtricitabine (FTC), entecavir (ETV), 2′,3′-dideoxyguanosine (ddG), 2′,3′-dideoxyadenosine (ddA), 2′-fluoro-2′,3′-dideoxyarabinosyladenine (F-ddA), or abacavir (ABC).

Embodiment 38. The LINE-1 inhibitor for use of Embodiment 37, wherein the LINE-1 inhibitor is islatravir.

Embodiment 39. The LINE-1 inhibitor for use of Embodiment 37, wherein the LINE-1 inhibitor is censavudine.

Embodiment 40. The LINE-1 inhibitor for use of any one of Embodiments 32-36, wherein the LINE-1 inhibitor is a compound of Formula I:

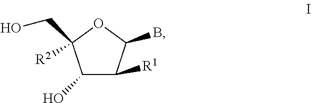

or a pharmaceutically acceptable salt or solvate thereof, or a tautomer thereof, wherein:
B is selected from the group consisting of:

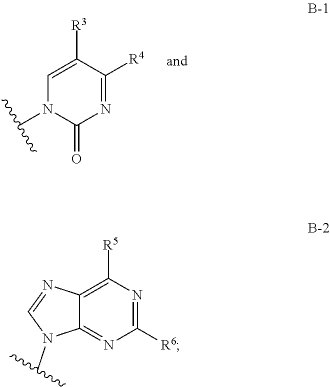

R¹ is selected from the group consisting of hydrogen and —OH;
R² is selected from the group consisting of methyl, ethynyl, and —CN;
R³ is selected from the group consisting of hydrogen, fluoro, chloro, bromo, iodo and methyl;
R⁴ is selected from the group consisting of —NH₂ and —OH;
R⁵ is selected from the group consisting of —NH₂ and —OH; and
R⁶ is selected from the group consisting of hydrogen, fluoro, chloro, and —NH₂.

Embodiment 41. The LINE-1 inhibitor for use of Embodiment 40, wherein the LINE-1 inhibitor is a compound of Formula II:

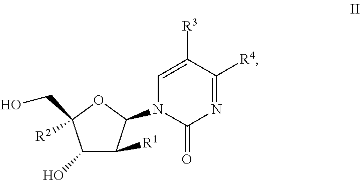

or a pharmaceutically acceptable salt or solvate thereof, or a tautomer thereof.

Embodiment 42. The LINE-1 inhibitor for use of Embodiment 41, wherein R³ is hydrogen.

Embodiment 43. The LINE-1 inhibitor for use of Embodiment 41, wherein R³ is selected from the group consisting of fluoro and chloro.

Embodiment 44. The LINE-1 inhibitor for use of Embodiment 41, wherein R³ is methyl.

Embodiment 45. The LINE-1 inhibitor for use of any one of Embodiments 41-44, wherein R⁴ is —NH₂.

Embodiment 46. The LINE-1 inhibitor for use of any one of Embodiments 41-44, wherein R⁴ is —OH.

Embodiment 47. The LINE-1 inhibitor for use of Embodiment 40, wherein the LINE-1 inhibitor is a compound of Formula III:

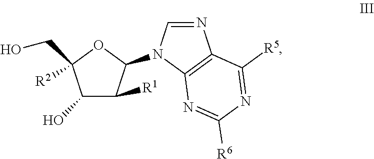

or a pharmaceutically acceptable salt or solvate thereof, or a tautomer thereof.

Embodiment 48. The LINE-1 inhibitor for use of Embodiment 47, wherein R⁵ is —NH₂.

Embodiment 49. The LINE-1 inhibitor for use of Embodiment 47, wherein R⁵ is —OH.

Embodiment 50. The LINE-1 inhibitor for use of any one of Embodiments 47-49, wherein R⁶ is hydrogen.

Embodiment 51. The LINE-1 inhibitor for use of any one of Embodiments 47-49, wherein R⁶ is chloro.

Embodiment 52. The LINE-1 inhibitor for use of any one of Embodiments 47-49, wherein R⁶ is fluoro.

Embodiment 53. The LINE-1 inhibitor for use of any one of Embodiments 47-49, wherein R⁶ is —NH₂.

Embodiment 54. The LINE-1 inhibitor for use of any one of Embodiments 40-53, wherein R¹ is hydrogen.

Embodiment 55. The LINE-1 inhibitor for use of any one of Embodiments 40-53, wherein R¹ is —OH.

Embodiment 56. The LINE-1 inhibitor for use of any one of Embodiments 40-55, wherein R² is methyl.

Embodiment 57. The LINE-1 inhibitor for use of any one of Embodiments 40-55, wherein R² is ethynyl.

Embodiment 58. The LINE-1 inhibitor for use of any one of Embodiments 40-55, wherein R² is —CN.

Embodiment 59. The LINE-1 inhibitor for use of Embodiment 40, wherein the LINE-1 inhibitor is a compound selected from the group consisting of.

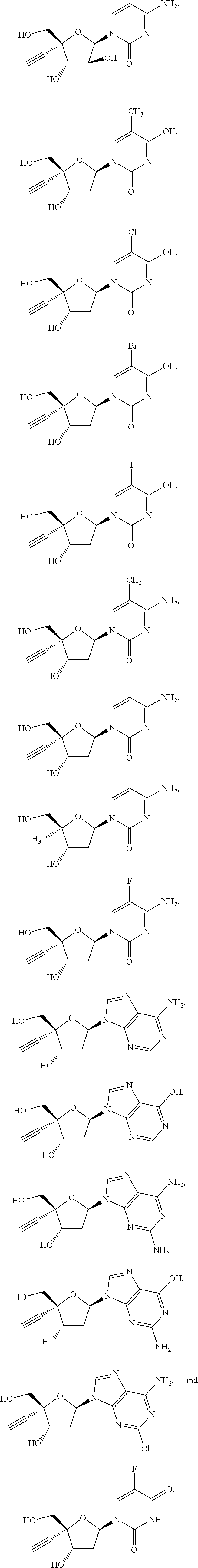

or a pharmaceutically acceptable salt or solvate thereof, or a tautomer thereof, or a compound of Table B, or a pharmaceutically acceptable salt or solvate thereof, or a tautomer thereof, or a compound of Table C, or a pharmaceutically acceptable salt or solvate thereof, or a tautomer thereof.

Embodiment 60. The LINE-1 inhibitor for use of any one of Embodiments 32-59, wherein the LINE-1 inhibitor is administered as a pharmaceutical composition comprising the LINE-1 inhibitor and pharmaceutically acceptable carrier.

Embodiment 61. The LINE-1 inhibitor for use of any one of Embodiments 32-60 further comprising administering one or more optional therapeutic agents to the subject.

Embodiment 62. The LINE-1 inhibitor for use of any one of Embodiments 32-61, wherein the subject is (a) not infected with the HIV virus, (b) is not suspected of being infected with the HIV virus, and/or (c) is not being treated to prevent infection with the HIV virus.

Embodiment 63. Use of a LINE-1 inhibitor in the manufacture of a medicament to enhance cognition, inhibit cognitive decline, treat or prevent a cognitive deficit disorder, or treat or prevent CJD in a subject in need thereof.

Embodiment 64. The use of Embodiment 63, wherein the LINE-1 inhibitor is administered to enhance cognition.

Embodiment 65. The use of Embodiment 63, wherein the LINE-1 inhibitor is administered to inhibit cognitive decline.

Embodiment 66. The use of Embodiment 63, wherein the LINE-1 inhibitor is administered to treat or prevent a cognitive deficit disorder.

Embodiment 67. The use of Embodiment 66, wherein the cognitive deficit disorder is a learning disorder, a memory disorder, a sensory perception disorder, an attention deficit/hyperactivity disorder, associated with autism or Asperger's syndrome, mild cognitive impairment, age-related cognitive decline, associated with traumatic, tumor-related, or ischemic brain injury, a drug-related cognitive impairment, or an alcohol-related cognitive impairment.

Embodiment 68. The use of any one of Embodiments 63-67, wherein the LINE-1 inhibitor is islatravir, censavudine, elvucitabine, lamivudine (3TC), zidovudine (AZT), tenofovir, tenofovir disoproxil, tenofovir alafenamide, stavudine (d4T), zalcitabine (ddC), didanosine (ddI), emtricitabine (FTC), entecavir (ETV), 2′,3′-dideoxyguanosine (ddG), 2′,3′-dideoxyadenosine (ddA), 2′-fluoro-2′,3′-dideoxyarabinosyladenine (F-ddA), or abacavir (ABC).

Embodiment 69. The use of Embodiment 68, wherein the LINE-1 inhibitor is islatravir.

Embodiment 70. The use of Embodiment 68, wherein the LINE-1 inhibitor is censavudine.

Embodiment 71. The use of any one of Embodiments 63-67, wherein the LINE-1 inhibitor is a compound of Formula I:

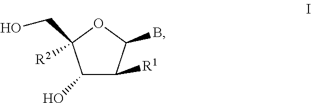

or a pharmaceutically acceptable salt or solvate thereof, or a tautomer thereof, wherein:
B is selected from the group consisting of:

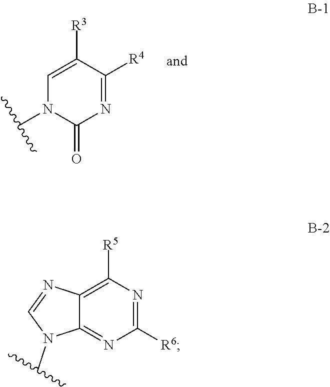

R¹ is selected from the group consisting of hydrogen and —OH;
R² is selected from the group consisting of methyl, ethynyl, and —CN;
R³ is selected from the group consisting of hydrogen, fluoro, chloro, bromo, iodo and methyl;
R⁴ is selected from the group consisting of —NH₂ and —OH;
R⁵ is selected from the group consisting of —NH₂ and —OH; and
R⁶ is selected from the group consisting of hydrogen, fluoro, chloro, and —NH₂.

Embodiment 72. The use of Embodiment 71, wherein the LINE-1 inhibitor is a compound of Formula II:

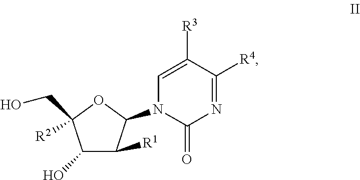

or a pharmaceutically acceptable salt or solvate thereof, or a tautomer thereof.

Embodiment 73. The use of Embodiment 72, wherein R³ is hydrogen.

Embodiment 74. The use of Embodiment 72, wherein R³ is selected from the group consisting of fluoro and chloro.

Embodiment 75. The use of Embodiment 72, wherein R³ is methyl.

Embodiment 76. The use of any one of Embodiments 72-75, wherein R⁴ is —NH₂.

Embodiment 77. The use of any one of Embodiments 72-75, wherein R⁴ is —OH.

Embodiment 78. The use of Embodiment 71, wherein the LINE-1 inhibitor is a compound of Formula III:

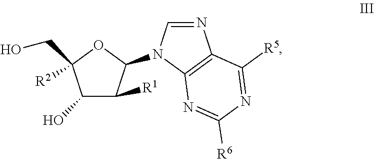

or a pharmaceutically acceptable salt or solvate thereof, or a tautomer thereof.

Embodiment 79. The use of Embodiment 78, wherein R⁵ is —NH₂.

Embodiment 80. The use of Embodiment 78, wherein R⁵ is —OH.

Embodiment 81. The use of any one of Embodiments 78-80, wherein R⁶ is hydrogen.

Embodiment 82. The use of any one of Embodiments 78-80, wherein R⁶ is chloro.

Embodiment 83. The use of any one of Embodiments 78-80, wherein R⁶ is fluoro.

Embodiment 84. The use of any one of Embodiments 78-80, wherein R⁶ is —NH₂.

Embodiment 85. The use of any one of Embodiments 71-84, wherein R¹ is hydrogen.

Embodiment 86. The use of any one of Embodiments 71-84, wherein R¹ is —OH.

Embodiment 87. The use of any one of Embodiments 71-86, wherein R² is methyl.

Embodiment 88. The use of any one of Embodiments 71-86, wherein R² is ethynyl.

Embodiment 89. The use of any one of Embodiments 71-86, wherein R² is —CN.

Embodiment 90. The use of Embodiment 71, wherein the LINE-1 inhibitor is a compound selected from the group consisting of.

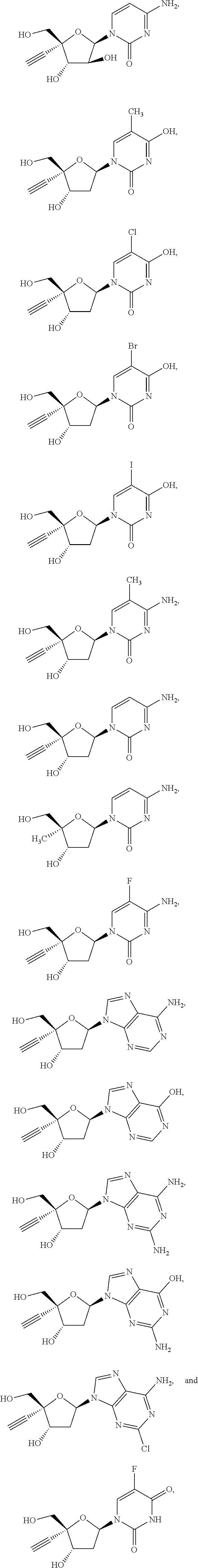

or a pharmaceutically acceptable salt or solvate thereof, or a tautomer thereof, or a compound of Table B, or a pharmaceutically acceptable salt or solvate thereof, or a tautomer thereof, or a compound of Table C, or a pharmaceutically acceptable salt or solvate thereof, or a tautomer thereof.

Embodiment 91. The use of any one of Embodiments 63-90, wherein the LINE-1 inhibitor is administered as a pharmaceutical composition comprising the LINE-1 inhibitor and pharmaceutically acceptable carrier.

Embodiment 92. The use of any one of Embodiments 63-91 further comprising administering one or more optional therapeutic agents to the subject.

Embodiment 93. The use of any one of Embodiments 63-92, wherein the subject is (a) not infected with the HIV virus, (b) is not suspected of being infected with the HIV virus, and/or (c) is not being treated to prevent infection with the HIV virus.

Embodiment 94. A kit comprising a LINE-1 inhibitor, or a pharmaceutical composition thereof, and instructions of administering the LINE-1 inhibitor, or pharmaceutical composition thereof, to enhance cognition, inhibit cognitive decline, or treat or prevent a cognitive deficit disorder in a subject in need thereof.

Embodiment 95. The method of any one of Embodiments 1 or 6-31, wherein the LINE-1 inhibitor is administered to treat or prevent CJD.

Embodiment 96. The LINE-1 inhibitor for use of any one of Embodiments 32 or 37-62, wherein the LINE-1 inhibitor is administered to treat or prevent CJD.

Embodiment 97. The use of any one of Embodiments 63 or 68-93, wherein the LINE-1 inhibitor is administered to treat or prevent CJD.

Embodiment 98. A kit comprising a LINE-1 inhibitor, or a pharmaceutical composition thereof, and instructions of administering the LINE-1 inhibitor, or pharmaceutical composition thereof, to treat or prevent CJD in a subject in need thereof.

### EXAMPLES

### Example 1

### Human LINE-1 Retrotransposition Assay

Islatravir and other compounds were tested for inhibition of retrotransposition activity of human LINE-1 in HeLa cells according to the following procedure.

HeLa cervical cancer cells were cultivated at 37° C. in a humidified 5% CO2 incubator in Dulbecco's Modified Eagle's Medium (DMEM)—high glucose, with 4500 mg/L glucose, L-glutamine, sodium pyruvate and sodium bicarbonate (Sigma), supplemented with 10% of heat inactivated fetal bovine serum (Thermo Fisher).

Assays were performed using reporter plasmid pYX017 as described (Xie, et al., 2011) with several modifications. The reporter assay was performed in 96-well white optical bottom plates. HeLa cells were seeded in wells 24 h prior to transfection and compound treatment so that cells were approximately 30% confluent on the day of transfection. Different cell plating densities were tested and a density of 2×10³ cells was determined to be optimal.

Compounds were resuspended in DMSO. Serial dilutions (1:3) were prepared in DMSO. Medium containing different concentrations of the compounds were prepared by adding 2 μl of the compound dilution to 1 ml of the culture medium. The final concentration of DMSO in the medium was 0.2%.

FuGENE® HD transfection reagent (Promega, E2311, Lot 382574 and Lot 397842) was used to transfect the plasmids into the cells. The transfection reagent: DNA mixture was prepared in OpiMEM (Thermo Fisher) according to manufacturer's instructions. Different ratios of transfection reagent to DNA were tested and a ratio of 3:1 was determined to be optimal. Culture medium was removed from the cells and discarded. The transfection reagent: DNA mixture (5 μl) was mixed with the compound containing medium (100 μl/well) and this was added onto the cells of each well. Cells were incubated at 37° C./5% C02 for different incubation time. A 72 h incubation time was determined to be optimal.

Luciferase reporter activity was quantified with the Dual-Luciferase® Reporter Assay System (Promega) according to manufacturer's instructions for multiwell plates except that cells were lysed directly on the multiwell plate with 30 μl of the passive lysis buffer (PLB) for 20 min at room temperature, with gentle shaking to ensure complete cell lysis.

Firefly and Renilla luciferase signals were measured using a SpectraMax i3x Multi-Mode Microplate Reader. Integration times of 100 ms and 10 ms were used to measure the Firefly and Renilla signals respectively. Relative L1 activity is calculated as Firefly/Renilla*1000 or Firefly/Renilla*10,000. Dose response inhibition data were fit to a four parameter logistic equation using non-linear regression (using Graphpad Prism 8), to determine IC₅₀ values for each inhibitor.

The results are provided in Table 1. Islatravir and censavudine exhibited unexpectedly better human LINE-1 inhibitor activity compared to the other reverse transcriptase inhibitory drugs tested.

TABLE 1
Human L1 activity inhibition

| | Human LINE-1 |
|---|---|
| Compound Name / No. | IC₅₀ (μM) |
| Lamivudine | 0.64 |
| Censavudine | 0.07 |
| Bictegravir | >12.41 |
| Efavirenz | >0.25 |
| Nevirapine | >50 |
| Rilpivirine | >15.08 |
| Zidovudine | 0.81 |
| Islatravir | 0.00129 |
| Raltegravir potassium | >9.86 |
| Dolutegravir sodium | >9.38 |
| Emtricitabine | 1.34 |
| Apricitabine | 6.46 |
| Tenofovir disoproxil | 0.2 |
| Tenofovir | 2.77 |
| Elvucitabine | 0.09 |
| Abacavir sulfate | 17.10 |
| Stavudine | 0.75 |
| Cpd. No. 2 | 0.3927 |
| Cpd. No. 4 | 0.489 |
| Cpd. No. 7 | 0.0091 |
| Cpd. No. 9 | 0.0209 |
| Cpd. No. 13 | 0.00051 |
| Cpd. No. 15 | 0.8333 |
| Cpd. No. 20 | 0.011 |
| Cpd. No. 23 | >50 |
| Cpd. No. 24 | 0.008 |
| Cpd. No. 26 | 2.0 |
| Cpd. No. 27 | 0.003 |

These results demonstrate that islatravir, censavudine, and compounds of Formulae I-ITT are surprisingly potent human LINE-1 reverse transcriptase inhibitors compared to the other reverse transcriptase inhibitors.

### Example 2

Compounds of Formula I-IIT can be prepared as described in Ohrui et al., J. Med. Chem. 43:4516-4525 (2000) and/or as described below for Cpd. No. 7.

### Synthesis of 4-amino-1-((2R,4S,5R)-5-ethynyl-4-hydroxy-5 (hydroxymethyl)tetrahydrofuran-2-yl)pyrimidin-2(1H)-one (Cpd. No. 7)

### Step 1: Synthesis of (2R,3S,5R)-5-(4-benzamido-2-oxopyrimidin-1(2H)-yl)-2-ethynyl-2-(((4-methylbenzoyl)oxy)methyl)tetrahydrofuran-3-yl 4-methylbenzoate and (2R,3S,5S)-5-(4-benzamido-2-oxopyrimidin-1(2H)-yl)-2-ethynyl-2-(((4-methylbenzoyl) oxy)methyl)tetrahydrofuran-3-yl 4-methylbenzoate

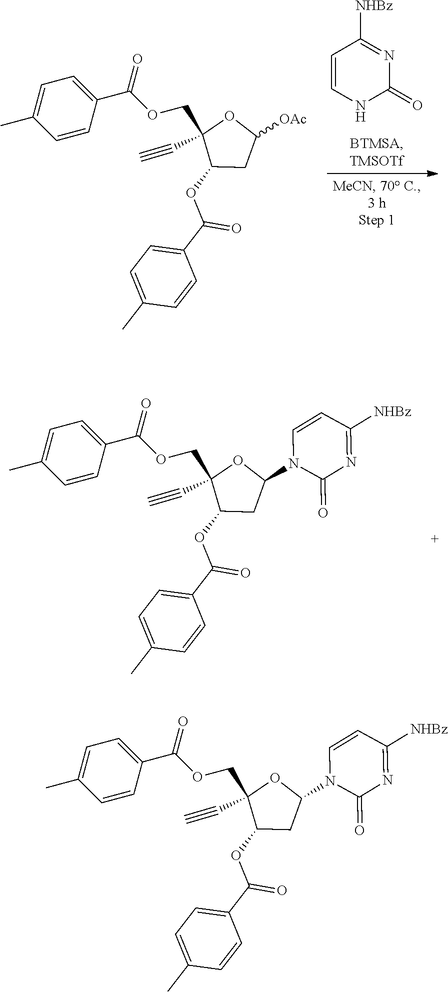

To a solution of N-(2-oxo-1H-pyrimidin-4-yl)benzamide (118 mg, 0.55 mmol) in MeCN (20 mL) was added BTMSA (234 mg, 1.37 mmol) at room temperature. The resulting mixture was heated at 70° C. for 1 h. After cooling to room temperature, TMSOTf (122 mg, 0.55 mmol) was added and the mixture was reheated to 70° C., then a solution of [(2R,3S)-5-acetoxy-2-ethynyl-3-(4-methylbenzoyl)oxy-tetrahydrofuran-2-yl]methyl4-methylbenzoate (200 mg, 0.46 mmol) in MeCN (5 mL) was added dropwise. After stirring at 70° C. for 2 h, the reaction mixture was poured into water (50 mL) and extracted with EtOAc (50 mL×2). The layers were separated, and the organic layer was concentrated. The residue was purified by prep-TLC eluting with 50% EtOAc in petroleum ether to give [(2R,3S,5R)-5-(4-benzamido-2-oxo-pyrimidin-1-yl)-2-ethynyl-3-(4-methylbenzoyl)oxy-tetrahydrofuran-2-yl]methyl 4-methylbenzoate (R_{f}=0.5) (60 mg, 22% yield) as a white solid and [(2R,3S,5S)-5-(4-benzamido-2-oxo-pyrimidin-1-yl)-2-ethynyl-3-(4-methyl benzoyl)oxy-tetrahydrofuran-2-yl]methyl 4-methylbenzoate (R_{f}=0.4) (60 mg, 22% yield) as a white solid.

### Step 2: Synthesis of 4-amino-1-((2R,4S,5R)-5-ethynyl-4-hydroxy-5 (hydroxymethyl)tetrahydrofuran-2-yl)pyrimidin-2(1H)-one

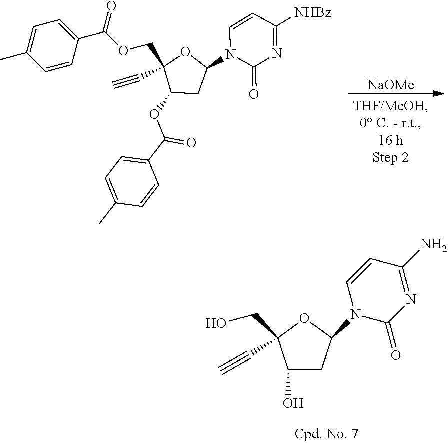

To a mixture of [(2R,3S,5R)-5-(4-benzamido-2-oxo-pyrimidin-1-yl)-2-ethynyl-3-(4-methylbenzoyl)oxy-tetrahydrofuran-2-yl]methyl4-methylbenzoate (60 mg, 0.1 mmol) in THE (5 mL) was added dropwise a solution of NaOMe (7 mg, 0.13 mmol) in MeOH (2 mL) at 0° C., then the resulting mixture was stirred at room temperature for 16 h. After that, the reaction mixture was concentrated under reduced pressure. The residue was purified by prep-HPLC to afford Cpd. No. 7 (8.8 mg, 34% yield) as a white solid. ¹H NMR (400 MHz, DMSO-d₆): δ 7.77 (d, J=7.2 Hz, 1H), 7.17-7.11 (m, 2H), 6.15-6.12 (m, 1H), 5.71 (d, J=7.2 Hz, 1H), 5.46 (s, 1H), 5.39 (s, 1H), 4.30-4.29 (m, 1H), 3.60-3.50 (m, 2H), 3.48 (s, 1H), 2.26-2.20 (m, 1H), 2.10-2.01 (m, 1H). LCMS (ESI): m/z 252.2 (M+H)⁺.

Having now fully described the compounds, methods, kits, and compositions herein, it will be understood by those of skill in the art that the same can be performed within a wide and equivalent range of conditions, formulations, and other parameters without affecting the scope of the methods, compounds, and compositions provided herein or any embodiment thereof. All patents, patent applications and publications cited herein are fully incorporated by reference herein in their entirety.

1. A method to enhance cognition, inhibit cognitive decline, treat or prevent a cognitive deficit disorder, or treat or prevent Creutzfeldt-Jakob disease (CJD) in a subject in need thereof, the method comprising administering to the subject a therapeutically effective amount of a LINE-1 inhibitor, wherein the LINE-1 inhibitor is censavudine,

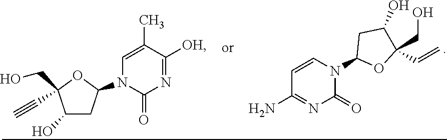 [SMILES: C#C[C@]1(CO)O[C@@H](n2cc(C)c(O)nc2=O)C[C@@H]1O ; "or" ; C=C[C@]1(CO)O[C@@H](n2ccc(N)nc2=O)C[C@@H]1O]

2. The method of claim 1, wherein the LINE-1 inhibitor is administered to enhance cognition.

3. The method of claim 1, wherein the LINE-1 inhibitor is administered to inhibit cognitive decline.

4. The method of claim 1, wherein the LINE-1 inhibitor is administered to treat or prevent a cognitive deficit disorder.

5. The method of claim 4, wherein the cognitive deficit disorder is a learning disorder, a memory disorder, a sensory perception disorder, an attention deficit/hyperactivity disorder, associated with autism or Asperger's syndrome, mild cognitive impairment, age-related cognitive decline, associated with traumatic, tumor-related, or ischemic brain injury, a drug-related cognitive impairment, or an alcohol-related cognitive impairment.

6. The method of claim 1, wherein the LINE-1 inhibitor is administered to treat or prevent Creutzfeldt-Jakob disease (CJD).

7.-8. (canceled)

9. The method of claim 1, wherein the LINE-1 inhibitor is censavudine.

10.-29. (canceled)

30. The method of claim 1, wherein the LINE-1 inhibitor is administered as a pharmaceutical composition comprising the LINE-1 inhibitor and pharmaceutically acceptable carrier.

31. The method of claim 1 further comprising administering one or more optional therapeutic agents to the subject.

32. The method of claim 1, wherein the subject is (a) not infected with the HIV virus, (b) is not suspected of being infected with the HIV virus, and/or (c) is not being treated to prevent infection with the HIV virus.

33. A kit comprising a LINE-1 inhibitor, or a pharmaceutical composition thereof, and instructions for administering the LINE-1 inhibitor, or pharmaceutical composition thereof, to enhance cognition, inhibit cognitive decline, treat or prevent a cognitive deficit disorder, or treat or prevent CJD in a subject in need thereof, wherein the LINE-1 inhibitor is censavudine,

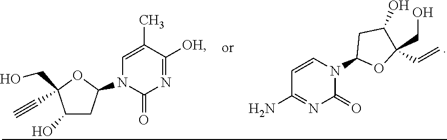 [SMILES: C#C[C@]1(CO)O[C@@H](n2cc(C)c(O)nc2=O)C[C@@H]1O ; "or" ; C=C[C@]1(CO)O[C@@H](n2ccc(N)nc2=O)C[C@@H]1O]

34. The method of claim 1, wherein the LINE-1 inhibitor is:

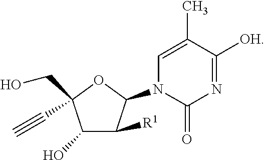

35. The method of claim 1, wherein the LINE-1 inhibitor is:

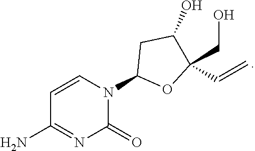 [SMILES: C=C[C@]1(CO)O[C@@H](n2ccc(N)nc2=O)C[C@@H]1O]

36. The kit of claim 33, wherein the LINE-1 inhibitor is censavudine.

37. The kit of claim 33, wherein the LINE-1 inhibitor is:

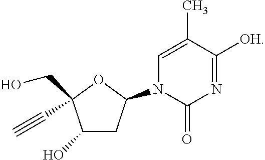 [SMILES: C#C[C@]1(CO)O[C@@H](n2cc(C)c(O)nc2=O)C[C@@H]1O]

38. The kit of claim 33, wherein the LINE-1 inhibitor is: 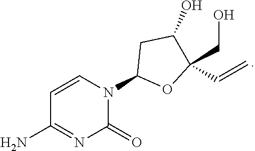 [SMILES: C=C[C@]1(CO)O[C@@H](n2ccc(N)nc2=O)C[C@@H]1O]
